(19) United States
(12) Patent Application Publication
(10) Pub. No.: US 2021/0214798 A1
(43) Pub. Date: Jul. 15, 2021

(54) METHODS AND DEVICES FOR DETECTING AND QUANTIFYING CELL-FREE DNA FRAGMENTS

(71) Applicant: Biological Dynamics, Inc., San Diego, CA (US)

(72) Inventors: Rajaram KRISHNAN, San Diego, CA (US); Juan Pablo HINESTROSA SALAZAR, San Diego, CA (US); Robert Paul TURNER, San Diego, CA (US); Robert KOVELMAN, La Jolla, CA (US)

(21) Appl. No.: 16/955,732

(22) PCT Filed: Dec. 19, 2018
(86) PCT No.: PCT/US2018/066605

§ 371 Date: Jun. 18, 2020

Related U.S. Application Data
(60) Provisional application No. 62/607,869, filed on Dec. 19, 2017.

(51) Int. Cl. C12Q 1/6886; C12Q 1/6806; C12Q 1/6883

(52) U.S. Cl. CPC C12Q 1/6886; C12Q 1/6883; C12Q 1/6806

(57) ABSTRACT

The present invention includes methods, devices and systems for isolating a nucleic acid from a fluid comprising cells. In various aspects, the methods, devices and systems may allow for a rapid procedure that requires a minimal amount of material and/or results in high purity nucleic acid isolated from complex fluids such as blood or environmental samples.

This patent application claims the benefit of U.S. Provisional Patent Application No. 62/607,869, filed Dec. 19, 2017, which is incorporated herein by reference in its entirety.

### BACKGROUND OF THE INVENTION

Progress has been made in detecting cancer using cell-free DNA. Methods such as next-generation sequencing aim to make access to cell-free DNA diagnostics more available. These advances will result in wider use of cell-free DNA diagnostics in cancer detection, prognostics, and therapy monitoring. However, current methods of analyzing cell-free DNA require significant expense, effort, and expertise to prepare and detect samples. New techniques and devices are needed that further simplify the analysis of cell-free DNA.

### SUMMARY OF THE INVENTION

The devices, methods, and kits disclosed herein fulfill a need for improved analysis of cell-free DNA. Some of the embodiments described herein can be used to isolate and analyze cell-free DNA from biological samples. In some embodiments, the devices and methods described herein can isolate, detect, quantify, and/or analyze cell-free DNA fragments of particular sizes. As will be described, the amounts of DNA in a sample corresponding to particular sizes or ranges of sizes can be used to detect, diagnose, classify, identify a disease or condition, determine a prognosis of a subject with a disease or condition, or evaluate the progress or efficacy of a treatment regimen for a subject with a disease or condition, including cancer.

Provided herein are methods for analyzing a biological sample from a subject. In some embodiments, the method comprises: capturing a plurality of nucleic acid fragments in the biological sample wherein the plurality of nucleic acid fragments comprises a plurality of sizes, wherein the plurality of sizes comprises nucleic acid fragments at least 250 bp in length; detecting the plurality of nucleic acids; and determining if the subject has a disease or condition based on the detection of nucleic acid fragments at least 250 in length.

In some embodiments, the nucleic acid fragments comprise cell-free DNA fragments and/or exosomes. In some embodiments, capturing the plurality of nucleic acid fragments comprises selectively capturing nucleic acid fragments between 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, 300-500 bp, 600-700 bp, 700-800 bp, 800-900 bp, 900-1000 bp, 1-2 kbp, 2-3 kbp, 3-4 kbp, 4-5 kbp, 5-6 kbp, 6-7 kbp, 7-8 kbp, 8-9 kbp, and/or 9-10 kbp in length.

In some embodiments, the disease or condition is cancer. In some embodiments, the disease or condition is an inflammatory disease, sepsis, heart disease, an alloimmune condition, or an autoimmune condition.

In some embodiments, the detecting comprises quantifying the plurality of nucleic acids. In some embodiments, quantifying the plurality of nucleic acids comprises quantifying an amount of nucleic acids that have a particular size or size range. In some embodiments, at least one size range comprises at least one size range of between 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, 300-500 bp, 600-700 bp, 700-800 bp, 800-900 bp, 900-1000 bp, 1-2 kbp, 2-3 kbp, 3-4 kbp, 4-5 kbp, 5-6 kbp, 6-7 kbp, 7-8 kbp, 8-9 kbp, and/or 9-10 kbp in length. In some embodiments, at least one size range comprises fragments that are at least 250 bp, 250 bp, 275 bp, 300 bp, 325 bp, 350 bp, 375 bp, 400 bp, 425 bp, 450 bp, 475 bp, 500 bp, 525 bp, 550 bp, 575 bp, 300 bp, 400 bp, 300 bp, 600 bp, 700 bp, 800 bp, 900 bp, 1 kbp, 2 kbp, 3 kbp, 4 kbp, 5 kbp, 6 kbp, 7 kbp, 8 kbp, 9 kbp, and/or 10 kbp in length.

In some embodiments, quantifying the plurality of nucleic acids comprises quantifying an amount of nucleic acids corresponding to at least one size. In some embodiments, the at least one size comprises at least one size of any whole number of base pairs between 250 bp and 10 kbp.

In some embodiments, the method further comprises comparing an amount of nucleic acids corresponding to a first size or size range to a second amount of nucleic acids corresponding to a second size or size range. In some embodiments, the method further comprises comparing the amount of nucleic acids to a control. In some embodiments, the method further comprises comparing an amount of nucleic acids corresponding to a first size or size range to a control. In some embodiments, the control is obtained from the subject at an earlier time, from the subject when the subject is presumed not to have cancer, from a healthy individual, from the subject before undergoing a treatment for cancer, from the subject when the subject was undergoing treatment for cancer, from the subject after undergoing treatment for cancer, or wherein the control is a value determined to be indicative of a subject with cancer or a subject without cancer.

In some embodiments, determining if the subject has cancer further comprises determining a type of cancer, a stage of cancer, a prognosis, a size of a tumor, a tumor burden, a change in an amount of cancer, a change in tumor size, a change in the number of tumors, a premalignant condition, or a precancerous condition.

In some embodiments, capturing the plurality of nucleic acid fragments comprises using an electrode configured to generate an AC dielectrophoretic field. In some embodiments, capturing the plurality of nucleic acid fragments comprises using a plurality of electrodes configured to generate a dielectrophoretic low field region and a dielectrophoretic high field region. In some embodiments, the dielectrophoretic low field region is produced using an alternating current having a voltage of 1 volt to 40 volts peak-peak; and/or a frequency of 5 Hz to 5,000,000 Hz, and duty cycles from 5% to 50%. In some embodiments, the dielectrophoretic high field region is produced using an alternating current having a voltage of 1 volt to 40 volts peak-peak; and/or a frequency of 5 Hz to 5,000,000 Hz, and duty cycles from 5% to 50%. In some embodiments, the plurality of electrodes are configured to selectively capture nucleic acid fragments between 250-600 bp, 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, 300-500 bp, 600-700 bp, 700-800 bp, 800-900 bp, 900-1000 bp, 1-2 kbp, 2-3 kbp, 3-4 kbp, 4-5 kbp, 5-6 kbp, 6-7 kbp, 7-8 kbp, 8-9 kbp, and/or 9-10 kbp in length.

In some embodiments, the plurality of electrodes is coated with a hydrogel. In some embodiments, the hydrogel comprises two or more layers of a synthetic polymer. In some embodiments, the hydrogel is spin-coated onto the electrodes. In some embodiments, the hydrogel has a viscosity between about 0.5 cP to about 5 cP prior to spin-coating. In some embodiments, the hydrogel has a thickness between about 0.1 microns and 1 micron.

In some embodiments, the electrode comprises a material selected from the group consisting of platinum, gold, aluminum, tantalum, gallium arsenide, copper, silver, brass, zinc, tin, nickel, silicon, palladium, titanium, graphite, carbon, and combinations thereof. In some embodiments, the electrode comprises a mixed-metal oxide. In some embodiments, the mixed-metal oxide is selected from the group consisting of titanium oxide, zirconium.

In some embodiments, the method further comprises detecting at least one analyte selected from the group consisting of RNA, nucleosomes, exosomes, extracellular vesicles, proteins, cell membrane fragments, mitochondria or cellular vesicles.

The method of any one of claims 1-29, wherein the biological sample comprises a bodily fluid, blood, serum, plasma, urine, saliva, cells, tissue, or a combination thereof.

The method of claim 30, wherein the biological sample comprises cells and the method further comprises lysing the cells. In some embodiments, the method further comprises eluting the captured nucleic acid fragments. In some embodiments, the method further comprises sequencing the nucleic acid fragments.

In some embodiments, an increase in the efficiency of capturing the DNA fragments increases the diagnostic or predictive power or accuracy of the method by at least 10%, 20%, 30%, 40%, 50%, 60%, 70%, 80%, 90%, 100%, 150%, 200%, 300%, 400%, 500%, 1000%. In some embodiments, an increase in the efficiency of capturing the DNA fragments decreases the false positive rate by at least 10%, 20%, 30%, 40%, 50%, 60%, 70%, 80%, 90%, 100%, 150%, 200%, 300%, 400%, 500%, 1000%. In some embodiments, an increase in the efficiency of capturing the DNA fragments decreases the false negative rate by at least 10%, 20%, 30%, 40%, 50%, 60%, 70%, 80%, 90%, 100%, 150%, 200%, 300%, 400%, 500%, 1000%. In some embodiments, the method performance is characterized by an area under the receiver operating characteristic (ROC) curve (AUC) ranging from 0.60 to 0.70, 0.70 to 0.79, 0.80 to 0.89, or 0.90 to 1.00.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the results of cfDNA samples from patients with adenocarcinoma, squamous cell cancer, and ovarian cancer, and a healthy control that were isolated on the described devices.

FIG. 2 shows a comparison of cfDNA concentrations for 52 healthy patients and 53 cancer patients (lung, breast, ovarian, and pancreatic cancers).

FIG. 3A and FIG. 3B show the concentration of cfDNA in two patients as they undergo treatment for cancer. The figures show the concentration of cdDNA on the Y axis as ng/mL and the X axis represents time.

### DETAILED DESCRIPTION OF THE INVENTION

Described herein are methods, devices and systems suitable for isolating or separating cellular components or molecules from a fluid composition. Examples of such components and molecules include DNA, including cell-free DNA and DNA fragments, RNA, nucleosomes, exosomes, extracellular vesicles, proteins, cell membrane fragments, mitochondria or cellular vesicles.

In specific embodiments, provided herein are methods, devices and systems for isolating or separating a nucleic acid, including cell-free nucleic acid, from a fluid comprising cells or other particulate material. In some embodiments, the methods, devices, and systems allow for quantification of the nucleic acids. In various aspects, the methods, devices and systems may allow for a rapid procedure that requires a minimal amount of material and/or results in high purity DNA isolated from complex fluids such as blood or environmental samples.

Cell-free DNA fragments can be predictive of cancer diagnoses in patients. Others have shown that cell-free DNA fragmentation correlates with tumor progression. As a result, patients with cancer have a higher proportion of shorter DNA fragments in their blood than patients without cancer. In one study, the concentration of fragments approximately 73 bp in size rose significantly in mice as tumors grew. In contrast, the concentrations of fragments approximately 145 bp rose only slightly as tumors grew and the concentration of fragments approximately 300 bp long ultimately went down as tumors grew (Mouliere et al. (2011) PLoS ONE 6(9): e23418.)

Contrary to studies like these, the inventors have surprisingly discovered that the amount of DNA fragments larger than 300 bp, for example, between 300 and 500 bp or larger, can be predictive of future risks of cancer. As a result, the tests described herein that detect larger DNA fragments, i.e., at least 300 bp, at least 400 bp, at least 500 bp or more, can be used for disease screening, early detection, and risk prediction, for example, in cancer and metastatic diseases. Other diseases or conditions are also contemplated. These include, for example, infectious diseases or conditions, sepsis, alloimmune and autoimmune diseases or conditions, including those related to transplant complications or rejection, inflammatory diseases or conditions, or heart disease or heart conditions. The simplicity and relatively low costs of performing the tests described herein can also allow for more frequent testing. As a result, some patients may be tested between one and six times a year, or more, depending on a variety of risk factors.

Patients can benefit from frequent, low-cost testing. For example, frequent testing allows patients and their healthcare providers to generate patient profiles using data from longitudinal studies. These profiles can be used to distinguish normal, healthy states from states that may indicate the presence of a disease or condition, such as cancer. The use of more frequent testing can also help patients detect diseases or conditions earlier. Thus, the use of more frequent testing has the potential to both increase the accuracy of the tests while also allowing for prognoses as patients begin treatment regimens earlier.

Provided in certain embodiments herein are methods, devices and systems for isolating or separating particles or molecules from a fluid composition, the methods, devices and systems comprising applying the fluid to a device and isolating nucleic acid from a sample of at least 300 bp. In some embodiments, the nucleic acid is quantified and compared against a standard sample. In other embodiments, the nucleic acid is analyzed for presence of at least one biomarker. In yet other embodiments, the nucleic acid is isolated as cell-free nucleic acid. In some embodiments, the nucleic acid is DNA. In yet other embodiments, the nucleic acid is RNA.

In some embodiments, the nucleic acid is isolated by centrifuging the plasma or serum. Such centrifugations can be accompanied by heat as described by method of Emanuel and Pestka (1993). Other methods include the use of a detergent, heat, and phenol, as described in Xue, Xiaoyan, et al. “Optimizing the yield and utility of circulating cell-free DNA from plasma and serum.” Clinica Chimica Acta 404.2 (2009): 100-104, incorporated herein by reference.

Other methods include the use of phenol chloroform extraction. In an exemplary method, plasma is treated with lx SDS/Proteinase K solution (0.5 mg/ml) (1:1), incubated overnight at 56° C. followed by phenol-chloroform (4:1) treatment and centrifuged at 7,000 rpm. The upper layer is transferred into fresh 15 ml centrifuge tubes and the same step is repeated again; DNA is precipitated by adding glycogen (0.1 μg/μl), ammonium acetate (7.5 M) and absolute alcohol. A DNA pellet is obtained by centrifuging at 7,000 rpm.

In some embodiments, the nucleic acid is isolated using a DNA extraction column, such as a QlAamp Blood Kit, QlAamp MinElute ccfDNA kit, or the EZ1 ccfDNA kit (Qiagen, Hilden, Germany). Other methods include the use of magnetic beads, including the use of MagMAX cell-free DNA isolation kits from ThermoFisher Scientific or cfPure kits from BioChain.

Provided in certain embodiments herein are methods, devices and systems for isolating or separating particles or molecules from a fluid composition, the methods, devices, and systems comprising applying the fluid to a device comprising an array of electrodes and being capable of generating AC electrokinetic forces (e.g., when the array of electrodes are energized). In some embodiments, the dielectrophoretic field, is a component of AC electrokinetic force effects. In other embodiments, the component of AC electrokinetic force effects is AC electroosmosis or AC electrothermal effects. In some embodiments the AC electrokinetic force, including dielectrophoretic fields, comprises high-field regions (positive DEP, i.e. area where there is a strong concentration of electric field lines due to a non-uniform electric field) and/or low-field regions (negative DEP, i.e. area where there is a weak concentration of electric field lines due to a non-uniform electric field).

In specific instances, the particles, cellular components, or molecules (e.g., nucleic acid) are isolated (e.g., isolated or separated from cells) in a field region (e.g., a high field region) of the dielectrophoretic field. In some embodiments, the particles, cellular components, or molecules are isolated from a bodily fluid, blood, serum, plasma, urine, saliva, cells, tissue, or a combination thereof. In some embodiments, the particles, cellular components, or molecules are isolated from a fluid or a portion of a fluid that is cell-free. In some embodiments, the particles, cellular components, or molecules are or comprise DNA, including cell-free DNA, RNA, nucleosomes, exosomes, extracellular vesicles, proteins, cell membrane fragments, mitochondria or cellular vesicles, as well as fragments or portions of any of the above.

In some embodiments, the method, device, or system further includes one or more of the following steps: concentrating particles or molecules of interest in a first dielectrophoretic field region (e.g., a high field DEP region), and removing contaminants from the particles or molecules of interest by flushing or washing the region. In other embodiments, the method, device, or system includes one or more of the following steps: concentrating cells and other residual material or contaminants in a first dielectrophoretic field region (e.g., a low field DEP region), concentrating nucleic acid in a second dielectrophoretic field region (e.g., a high field DEP region), and washing away the cells and residual material. The method also optionally includes devices and/or systems capable of performing one or more of the following steps: washing or otherwise removing residual (e.g., cellular) material from the nucleic acid (e.g., rinsing the array with water or buffer while the nucleic acid is concentrated and maintained within a high field DEP region of the array), degrading residual proteins (e.g., residual proteins from lysed cells and/or other sources, such degradation occurring according to any suitable mechanism, such as with heat, a protease, or a chemical), flushing degraded proteins from the nucleic acid, and collecting the nucleic acid. In some embodiments, the result of the methods, operation of the devices, and operation of the systems described herein is an isolated nucleic acid, optionally of suitable quantity and purity for DNA sequencing. In some embodiments, the particles or molecules of interest is DNA, including cell-free DNA and DNA fragments, RNA, including cell-free RNA, nucleosomes, exosomes, extracellular vesicles, proteins (including proteins expressed on the surface of endosomes), protein fragments, cell membrane fragments, mitochondria, cellular vesicles, and vesicles of endosomal origin.

In some instances, it is advantageous that the methods described herein are performed in a short amount of time, the devices are operated in a short amount of time, and the systems are operated in a short amount of time. In some embodiments, the period of time is short with reference to the “procedure time” measured from the time between adding the fluid to the device and obtaining isolated nucleic acid. In some embodiments, the procedure time is less than 3 hours, less than 2 hours, less than 1 hour, less than 30 minutes, less than 20 minutes, less than 10 minutes, or less than 5 minutes.

In another aspect, the period of time is short with reference to the “hands-on time” measured as the cumulative amount of time that a person must attend to the procedure from the time between adding the fluid to the device and obtaining isolated nucleic acid. In some embodiments, the hands-on time is less than 20 minutes, less than 10 minutes, less than 5 minute, less than 1 minute, or less than 30 seconds.

In some instances, it is advantageous that the devices described herein comprise a single vessel, the systems described herein comprise a device comprising a single vessel and the methods described herein can be performed in a single vessel, e.g., in a dielectrophoretic device as described herein. In some aspects, such a single-vessel embodiment minimizes the number of fluid handling steps and/or is performed in a short amount of time. In some instances, the present methods, devices and systems are contrasted with methods, devices and systems that use one or more centrifugation steps and/or medium exchanges. In some instances, centrifugation increases the amount of hands-on time required to isolate nucleic acids. In another aspect, the single-vessel procedure or device isolates nucleic acids using a minimal amount of consumable reagents.

### Devices and Systems

In some embodiments, described herein are devices for collecting cellular material from a fluid. In one aspect, described herein are devices for collecting a cellular material from a fluid comprising cells, from a cell-free portion of a fluid, or other particulate material.

In some embodiments, disclosed herein are devices and methods for isolating particles or molecules of interest from a biological or environmental sample, and analyzing the particles or molecules of interest for nucleic acid sequences of interest. In some embodiments, the devices and methods disclosed herein identify subjects or patients with a disease. Examplary dieases include cancer, such as breast cancer, lung cancer (including non-small cell lung cancer, including adenocarcinoma, and small cell lung cancer), colorectal cancer, pancreatic cancer, and ovarian cancer. The diseases can also include inflammatory diseases.

In other embodiments, the methods and devices disclosed herein identify subjects or patients with a disease by detecting specific biomarkers. Exemplary biomarkers include nucleic acids, such as nucleic acids and fragments therof of varying sizes, and proteins including but not limited to PD-Ll and carcinoembrionic antigen (CEA). Protein markers for detection using the methods described herein include, but are not limited to, carcinoembryonic antigen (CEA), CA125, CA27.29, CA15-3, CA19.9, Prolactin, Ki-67, estrogen receptor alpha, CD30, CD30L, CD10, surviving, AZU1, alpha-fetoprotein (aFP), (3-human chorionic gonadotropin. (βHCG), glypican-1 (GPC-1), CYFRA-21, RNA-based markers and prostate specific antigen (PSA).

Additional cancer markers that may be detecting using the methods described herein include, but are not limited to, BRAF, BRCA1, BRCA2, CD20, Calcitonin, Calretinin, CD34, CD99MIC 2, CD117, Chromogranin, Cytokeratin (various types), Desmin, Epithelial membrane antigen (EMA), Factor VIII, CD31 FL1, Glial fibrillary acidic protein (GFAP), Gross cystic disease fluid protein (GCDFP-15), HER2/neu, HER3, HMB-45, Human chorionic gonadotropin (hCG), inhibin, keratin (various types), lymphocyte marker, MART-1 (Melan-A), Mesothelin, Myo D1, MUC-1, MUC-16 neuron-specific enolase (NSE), placental alkaline phosphatase (PLAP), leukocyte common antigen (CD45), S100 protein, synaptophysin, thyroglobulin, thyroid transcription factor-1, Tumor M2-PK, and vimentin.

Additional markers of inflammation that may be detecting using the methods described herein include, but are not limited to, Carcinoembryonic antigen (CEA), plasma a-fetoprotein (αFP), β human chorionic gonadotrophin (βHCG), C-reactive protein (CRP), Lysosome granules, Histamine, IFN-gamma, Interleukin (IL)-8, Leukotriene B4, Nitric oxide, Prostaglandins, TNF-α, and IL-1.

Cardiac markers include Creatine Kinase (CKMB), Myoglobin and Troponin 1. Markers for anemia include Ferritin. Metabolic markers include Cortisol (CORT) and Human Growth Hormone (HGH). Kidney markers include Cystatin C (CysC), β₂ Microglobulin (BMG), intact Parathyroid Hormone (iPTH). Diabetes markers include C-peptide, Glycated Homoglobin (HbAlc) and Insulin (IRI). Thyroid hormone markers include Tyroid-Stimulating Hormone (TSH) while reproductive hormone markers include (3HCG, Follicle-stimulating hormone (FSH), Luteinizing Hormone II (LH II) and Prolactatin (PRL).

In yet other embodiments, the methods and devices disclosed herein identify subject or patients with a disease by quantifying the particles or molecules of interest, including nucleic acid, RNA, DNA, nucleosomes, exosomes, extracellular vesicles, proteins, cell membrane fragments, mitochondria or cellular vesicles. In still other embodiments, the methods and devices disclosed herein determine the prognosis of a patient or subject during treatment of the identified disease.

In some embodiments, disclosed herein is a device for isolating cellular material, the device comprising a centrifuge. In some embodiments, the device can also comprise robotic elements configured to add and remove fluids from columns on the centrifuge. In some embodiments, the device further comprises a heater. In some embodiments, the device further comprises fluid reservoirs for storing and dispensing buffers and solutions used to isolate, purify, or elute nucleic acids from the columns.

In some embodiments, disclosed herein is a device for isolating cellular material, the device comprising: a. a housing; b. a heater or thermal source and/or a reservoir comprising a protein degradation agent; and c. a plurality of alternating current (AC) electrodes within the housing, the AC electrodes configured to be selectively energized to establish AC electrokinetic high field and AC electrokinetic low field regions, whereby AC electrokinetic effects provide for concentration of cells in low field regions of the device. In some embodiments, the plurality of electrodes is configured to be selectively energized to establish a dielectrophoretic high field and dielectrophoretic low field regions. In some embodiments, the protein degradation agent is a protease. In some embodiments, the protein degradation agent is Proteinase K. In some embodiments, the device further comprises a second reservoir comprising an eluant.

In some embodiments, disclosed herein is a device comprising: a. a plurality of alternating current (AC) electrodes, the AC electrodes configured to be selectively energized to establish AC electrokinetic high field and AC electrokinetic low field regions; and b. a module capable of thermocycling and performing PCR or other enzymatic reactions.

In some embodiments, disclosed herein is a device comprising: a. a plurality of alternating current (AC) electrodes, the AC electrodes configured to be selectively energized to establish AC electrokinetic high field and AC electrokinetic low field regions; and b. a module capable of imaging the material captured or isolated by the AC electrodes. Some embodiments also include chambers and fluidics for adding reagents and removing that allow for the visualization of the captured materials.

In some embodiments, the plurality of electrodes is configured to be selectively energized to establish a dielectrophoretic high field and dielectrophoretic low field regions. In some embodiments, the device is capable of isolating DNA, including cell-free DNA and DNA fragments, RNA, nucleosomes, exosomes, extracellular vesicles, proteins, cell membrane fragments, mitochondria and cellular vesicles from a biological sample comprising fluid. In some embodiments, the device is capable of isolating these materials from cells in the biological sample.

In some embodiments, the device is configured to preferentially isolate, separate, sort, or select for one or more materials from the biological sample. In some embodiments, the device is configured to preferentially isolate, separate, sort, or select for one or more materials from the biological sample based on size. In some embodiments, the device is configured to detect the one or more materials from the biological sample.

In some embodiments, the device is capable of performing PCR amplification or other enzymatic reactions. In some embodiments, DNA is isolated and PCR or other enzymatic reaction is performed in a single chamber. In some embodiments, DNA is isolated and PCR or other enzymatic reaction is performed in multiple regions of a single chamber. In some embodiments, DNA is isolated and PCR or other enzymatic reaction is performed in multiple chambers.

In some embodiments, the device further comprises at least one of an elution tube, a chamber and a reservoir to perform PCR amplification or other enzymatic reaction. In some embodiments, PCR amplification or other enzymatic reaction is performed in a serpentine microchannel comprising a plurality of temperature zones. In some embodiments, PCR amplification or other enzymatic reaction is performed in aqueous droplets entrapped in immiscible fluids (i.e., digital PCR). In some embodiments, the thermocycling comprises convection. In some embodiments, the device comprises a surface contacting or proximal to the electrodes, wherein the surface is functionalized with biological ligands that are capable of selectively capturing biomolecules.

In some embodiments, disclosed herein is a system for isolating a cellular material from a biological sample, the system comprising: a. a device comprising a plurality of alternating current (AC) electrodes, the AC electrodes configured to be selectively energized to establish AC electrokinetic high field and AC electrokinetic low field regions, whereby AC electrokinetic effects provide for concentration of cells in high field regions of the device; and b. a sequencer, thermocycler or other device for performing enzymatic reactions on isolated or collected nucleic acid. In some embodiments, the plurality of electrodes is configured to be selectively energized to establish a dielectrophoretic high field and dielectrophoretic low field regions.

In various embodiments, DEP fields are created or capable of being created by selectively energizing an array of electrodes as described herein. The electrodes are optionally made of any suitable material resistant to corrosion, including metals, such as noble metals (e.g. platinum, platinum iridium alloy, palladium, gold, and the like). In various embodiments, electrodes are of any suitable size, of any suitable orientation, of any suitable spacing, energized or capable of being energized in any suitable manner, and the like such that suitable DEP and/or other electrokinetic fields are produced.

In some embodiments described herein are methods, devices and systems in which the electrodes are placed into separate chambers and positive DEP regions and negative DEP regions are created within an inner chamber by passage of the AC DEP field through pore or hole structures. Various geometries are used to form the desired positive DEP (high field) regions and DEP negative (low field) regions for carrying cellular, microparticle, nanoparticle, and nucleic acid separations. In some embodiments, pore or hole structures contain (or are filled with) porous material (hydrogels) or are covered with porous membrane structures. In some embodiments, by segregating the electrodes into separate chambers, such pore/hole structure DEP devices reduce electrochemistry effects, heating, or chaotic fluidic movement from occurring in the inner separation chamber during the DEP process.

In one aspect, described herein is a device comprising electrodes, wherein the electrodes are placed into separate chambers and DEP fields are created within an inner chamber by passage through pore structures. The exemplary device includes a plurality of electrodes and electrode-containing chambers within a housing. A controller of the device independently controls the electrodes, as described further in PCT patent publication WO 2009/146143 A2, which is incorporated herein for such disclosure.

In some embodiments, chambered devices are created with a variety of pore and/or hole structures (nanoscale, microscale and even macroscale) and contain membranes, gels or filtering materials which control, confine or prevent cells, nanoparticles or other entities from diffusing or being transported into the inner chambers while the AC/DC electric fields, solute molecules, buffer and other small molecules can pass through the chambers.

In various embodiments, a variety of configurations for the devices are possible. For example, a device comprising a larger array of electrodes, for example in a square or rectangular pattern configured to create a repeating non-uniform electric field to enable AC electrokinetics. For illustrative purposes only, a suitable electrode array may include, but is not limited to, a 10×10 electrode configuration, a 50×50 electrode configuration, a 10×100 electrode configuration, 20×100 electrode configuration, or a 20×80 electrode configuration.

Such devices include, but are not limited to, multiplexed electrode and chambered devices, devices that allow reconfigurable electric field patterns to be created, devices that combine DC electrophoretic and fluidic processes; sample preparation devices, sample preparation, enzymatic manipulation of isolated nucleic acid molecules and diagnostic devices that include subsequent detection and analysis, lab-on-chip devices, point-of-care and other clinical diagnostic systems or versions.

In some embodiments, a planar platinum electrode array device comprises a housing through which a sample fluid flows. In some embodiments, fluid flows from an inlet end to an outlet end, optionally comprising a lateral analyte outlet. The exemplary device includes multiple AC electrodes. In some embodiments, the sample consists of a combination of micron-sized entities or cells, larger nanoparticulates and smaller nanoparticulates or biomolecules. In some instances, the larger nanoparticulates are cellular debris dispersed in the sample. In some embodiments, the smaller nanoparticulates are proteins, smaller DNA, RNA and cellular fragments. In some embodiments, the planar electrode array device is a 60×20 electrode array that is optionally sectioned into three 20×20 arrays that can be separately controlled but operated simultaneously. The optional auxiliary DC electrodes can be switched on to positive charge, while the optional DC electrodes are switched on to negative charge for electrophoretic purposes. In some instances, each of the controlled AC and DC systems is used in both a continuous and/or pulsed manner (e.g., each can be pulsed on and off at relatively short time intervals) in various embodiments. The optional planar electrode arrays along the sides of the sample flow, when over-layered with nanoporous material (e.g., a hydrogel of synthetic polymer), are optionally used to generate DC electrophoretic forces as well as AC DEP. Additionally, microelectrophoretic separation processes is optionally carried out within the nanopore layers using planar electrodes in the array and/or auxiliary electrodes in the x-y-z dimensions.

In various embodiments these methods, devices and systems are operated in the AC frequency range of from 1,000 Hz to 100 MHz, at voltages which could range from approximately 1 volt to 2000 volts pk-pk; at DC voltages from 1 volt to 1000 volts, at flow rates of from 10 microliters per minute to 10 milliliter per minute, and in temperature ranges from 1° C. to 120° C. In some embodiments, the methods, devices and systems are operated in AC frequency ranges of from about 3 to about 15 kHz. In some embodiments, the methods, devices, and systems are operated at voltages of from 5-25 volts pk-pk. In some embodiments, the methods, devices and systems are operated at voltages of from about 1 to about 50 volts/cm. In some embodiments, the methods, devices and systems are operated at DC voltages of from about 1 to about 5 volts. In some embodiments, the methods, devices and systems are operated at a flow rate of from about 10 microliters to about 500 microliters per minute. In some embodiments, the methods, devices and systems are operated in temperature ranges of from about 20° C. to about 60° C. In some embodiments, the methods, devices and systems are operated in AC frequency ranges of from 1,000 Hz to 10 MHz. In some embodiments, the methods, devices and systems are operated in AC frequency ranges of from 1,000 Hz to 1 MHz. In some embodiments, the methods, devices and systems are operated in AC frequency ranges of from 1,000 Hz to 100 kHz. In some embodiments, the methods, devices and systems are operated in AC frequency ranges of from 1,000 Hz to 10 kHz. In some embodiments, the methods, devices and systems are operated in AC frequency ranges of from 10 kHz to 100 kHz. In some embodiments, the methods, devices and systems are operated in AC frequency ranges of from 100 kHz to 1 MHz. In some embodiments, the methods, devices and systems are operated at voltages from approximately 1 volt to 1500 volts pk-pk. In some embodiments, the methods, devices and systems are operated at voltages from approximately 1 volt to 1500 volts pk-pk. In some embodiments, the methods, devices and systems are operated at voltages from approximately 1 volt to 1000 volts pk-pk. In some embodiments, the methods, devices and systems are operated at voltages from approximately 1 volt to 500 volts pk-pk. In some embodiments, the methods, devices and systems are operated at voltages from approximately 1 volt to 250 volts pk-pk. In some embodiments, the methods, devices and systems are operated at voltages from approximately 1 volt to 100 volts pk-pk. In some embodiments, the methods, devices and systems are operated at voltages from approximately 1 volt to 50 volts pk-pk. In some embodiments, the methods, devices and systems are operated at DC voltages from 1 volt to 1000 volts. In some embodiments, the methods, devices and systems are operated at DC voltages from 1 volt to 500 volts. In some embodiments, the methods, devices and systems are operated at DC voltages from 1 volt to 250 volts. In some embodiments, the methods, devices and systems are operated at DC voltages from 1 volt to 100 volts. In some embodiments, the methods, devices and systems are operated at DC voltages from 1 volt to 50 volts. In some embodiments, the methods, devices, and systems are operated at flow rates of from 10 microliters per minute to 1 ml per minute. In some embodiments, the methods, devices, and systems are operated at flow rates of from 10 microliters per minute to 500 microliters per minute. In some embodiments, the methods, devices, and systems are operated at flow rates of from 10 microliters per minute to 250 microliters per minute. In some embodiments, the methods, devices, and systems are operated at flow rates of from 10 microliters per minute to 100 microliters per minute. In some embodiments, the methods, devices, and systems are operated in temperature ranges from 1° C. to 100° C. In some embodiments, the methods, devices, and systems are operated in temperature ranges from 20° C. to 95° C. In some embodiments, the methods, devices, and systems are operated in temperature ranges from 25° C. to 100° C. In some embodiments, the methods, devices, and systems are operated at room temperature.

In some embodiments, the controller independently controls each of the electrodes. In some embodiments, the controller is externally connected to the device such as by a socket and plug connection, or is integrated with the device housing.

Also described herein are scaled sectioned (x-y dimensional) arrays of robust electrodes and strategically placed (x-y-z dimensional) arrangements of auxiliary electrodes that combine DEP, electrophoretic, and fluidic forces, and use thereof. In some embodiments, clinically relevant volumes of blood, serum, plasma, or other samples are more directly analyzed under higher ionic strength and/or conductance conditions. Described herein is the overlaying of robust electrode structures (e.g. platinum, palladium, gold, etc.) with one or more porous layers of materials (natural or synthetic porous hydrogels, membranes, controlled nanopore materials, and thin dielectric layered materials) to reduce the effects of any electrochemistry (electrolysis) reactions, heating, and chaotic fluid movement that may occur on or near the electrodes, and still allow the effective separation of cells, bacteria, virus, nanoparticles, DNA, and other biomolecules to be carried out. In some embodiments, in addition to using AC frequency cross-over points to achieve higher resolution separations, on-device (on-array) DC microelectrophoresis is used for secondary separations. For example, the separation of DNA nanoparticulates (20-50 kb), high molecular weight DNA (5-20 kb), intermediate molecular weight DNA (1-5 kb), and lower molecular weight DNA (0.1 -1kb) fragments may be accomplished through DC microelectrophoresis on the array. In some embodiments, the device is sub-sectioned, optionally for purposes of concurrent separations of different blood cells, bacteria and virus, and DNA carried out simultaneously on such a device.

In some embodiments, the device comprises a housing and a heater or thermal source and/or a reservoir comprising a protein degradation agent. In some embodiments, the heater or thermal source is capable of increasing the temperature of the fluid to a desired temperature (e.g., to a temperature suitable for degrading proteins, about 30° C., 40° C., 50° C., 60° C., 70° C., or the like). In some embodiments, the heater or thermal source is suitable for operation as a PCR thermocycler. IN other embodiments, the heater or thermal source is used to maintain a constant temperature (isothermal conditions). In some embodiments, the protein degradation agent is a protease. In other embodiments, the protein degradation agent is Proteinase K and the heater or thermal source is used to inactivate the protein degradation agent.

In some embodiments, the device also comprises a plurality of alternating current (AC) electrodes within the housing, the AC electrodes capable of being configured to be selectively energized to establish dielectrophoretic (DEP) high field and dielectrophoretic (DEP) low field regions, whereby AC electrokinetic effects provide for concentration of cells in low field regions of the device. In some embodiments, the electrodes are selectively energized to provide the first AC electrokinetic field region and subsequently or continuously selectively energized to provide the second AC electrokinetic field region. For example, further description of the electrodes and the concentration of cells in DEP fields is found in PCT patent publication WO 2009/146143 A2, which is incorporated herein for such disclosure.

In some embodiments, the device comprises a second reservoir comprising an eluant. The eluant is any fluid suitable for eluting the isolated cellular material from the device. In some instances the eluant is water or a buffer. In some instances, the eluant comprises reagents required for a DNA sequencing method.

In some embodiments, the device comprises a plurality of reservoirs, each reservoir containing a reagents useful in the staining and washing of the isolated cellular material in the device. Examples include antibodies, oligonucleotides, probes, and dyes, buffers, washes, water, detergents, and solvents.

Also provided herein are systems and devices comprising a plurality of alternating current (AC) electrodes, the AC electrodes configured to be selectively energized to establish dielectrophoretic (DEP) high field and dielectrophoretic (DEP) low field regions. In some instances, AC electrokinetic effects provide for concentration of cells in low field regions and/or concentration (or collection or isolation) of molecules (e.g., macromolecules, such as nucleic acid) in high field regions of the DEP field.

Also provided herein are systems and devices comprising a pluarilty of direct current (DC) electrodes. In some embodiments, the plurality of DC electrodes comprises at least two rectangular electrodes, spread throughout the array. In some embodiments, the electrodes are located at the edges of the array. In some embodiments, DC electrodes are interspersed between AC electrodes.

In some embodiments, a system or device described herein comprises a means for manipulating nucleic acid. In some embodiments, a system or device described herein includes a means of performing enzymatic reactions. In other embodiments, a system or device described herein includes a means of performing polymerase chain reaction, isothermal amplification, ligation reactions, restriction analysis, nucleic acid cloning, transcription or translation assays, or other enzymatic-based molecular biology assay.

In some embodiments, a system or device described herein comprises a nucleic acid sequencer. The sequencer is optionally any suitable DNA sequencing device including but not limited to a Sanger sequencer, pyro-sequencer, ion semiconductor sequencer, polony sequencer, sequencing by ligation device, DNA nanoball sequencing device, or single molecule sequencing device.

In some embodiments, a system or device described herein is capable of maintaining a constant temperature. In some embodiments, a system or device described herein is capable of cooling the array or chamber. In some embodiments, a system or device described herein is capable of heating the array or chamber. In some embodiments, a system or device described herein comprises a thermocycler. In some embodiments, the devices disclosed herein comprises a localized temperature control element. In some embodiments, the devices disclosed herein are capable of both sensing and controlling temperature.

In some embodiments, the devices further comprise heating or thermal elements. In some embodiments, a heating or thermal element is localized underneath an electrode. In some embodiments, the heating or thermal elements comprise a metal. In some embodiments, the heating or thermal elements comprise tantalum, aluminum, tungsten, or a combination thereof. Generally, the temperature achieved by a heating or thermal element is proportional to the current running through it. In some embodiments, the devices disclosed herein comprise localized cooling elements. In some embodiments, heat resistant elements are placed directly under the exposed electrode array. In some embodiments, the devices disclosed herein are capable of achieving and maintaining a temperature between about 20° C. and about 120° C. In some embodiments, the devices disclosed herein are capable of achieving and maintaining a temperature between about 30° C. and about 100° C. In other embodiments, the devices disclosed herein are capable of achieving and maintaining a temperature between about 20° C. and about 95° C. In some embodiments, the devices disclosed herein are capable of achieving and maintaining a temperature between about 25° C. and about 90° C., between about 25° C. and about 85° C., between about 25° C. and about 75° C., between about 25° C. and about 65° C. or between about 25° C. and about 55° C. In some embodiments, the devices disclosed herein are capable of achieving and maintaining a temperature of about 20° C., about 30° C., about 40° C., about 50° C., about 60° C., about 70° C., about 80° C., about 90° C., about 100° C., about 110° C. or about 120° C.

### Electrodes

The plurality of alternating current electrodes are optionally configured in any manner suitable for the separation processes described herein. For example, further description of the system or device including electrodes and/or concentration of cells in DEP fields is found in PCT patent publication WO 2009/146143, which is incorporated herein for such disclosure.

In some embodiments, the electrodes disclosed herein can comprise any suitable metal. In some embodiments, the electrodes can include but are not limited to: aluminum, copper, carbon, iron, silver, gold, palladium, platinum, iridium, platinum iridium alloy, ruthenium, rhodium, osmium, tantalum, titanium, tungsten, polysilicon, and indium tin oxide, or combinations thereof, as well as silicide materials such as platinum silicide, titanium silicide, gold silicide, or tungsten silicide. In some embodiments, the electrodes can comprise a conductive ink capable of being screen-printed.

In some embodiments, the edge to edge (E2E) to diameter ratio of an electrode is about 0.5 mm to about 5 mm. In some embodiments, the E2E to diameter ratio is about 1 mm to about 4 mm. In some embodiments, the E2E to diameter ratio is about 1 mm to about 3 mm. In some embodiments, the E2E to diameter ratio is about 1 mm to about 2 mm. In some embodiments, the E2E to diameter ratio is about 2 mm to about 5 mm. In some embodiments, the E2E to diameter ratio is about 1 mm. In some embodiments, the E2E to diameter ratio is about 2 mm. In some embodiments, the E2E to diameter ratio is about 3 mm. In some embodiments, the E2E to diameter ratio is about 4 mm. In some embodiments, the E2E to diameter ratio is about 5 mm.

In some embodiments, the electrodes disclosed herein are dry-etched. In some embodiments, the electrodes are wet etched. In some embodiments, the electrodes undergo a combination of dry etching and wet etching.

In some embodiments, each electrode is individually site-controlled.

In some embodiments, an array of electrodes is controlled as a unit.

In some embodiments, a passivation layer is employed. In some embodiments, a passivation layer can be formed from any suitable material known in the art. In some embodiments, the passivation layer comprises silicon nitride. In some embodiments, the passivation layer comprises silicon dioxide. In some embodiments, the passivation layer has a relative electrical permittivity of from about 2.0 to about 8.0. In some embodiments, the passivation layer has a relative electrical permittivity of from about 3.0 to about 8.0, about 4.0 to about 8.0 or about 5.0 to about 8.0. In some embodiments, the passivation layer has a relative electrical permittivity of about 2.0 to about 4.0. In some embodiments, the passivation layer has a relative electrical permittivity of from about 2.0 to about 3.0. In some embodiments, the passivation layer has a relative electrical permittivity of about 2.0, about 2.5, about 3.0, about 3.5 or about 4.0 .

In some embodiments, the passivation layer is between about 0.1 microns and about 10 microns in thickness. In some embodiments, the passivation layer is between about 0.5 microns and 8 microns in thickness. In some embodiments, the passivation layer is between about 1.0 micron and 5 microns in thickness. In some embodiments, the passivation layer is between about 1.0 micron and 4 microns in thickness. In some embodiments, the passivation layer is between about 1.0 micron and 3 microns in thickness. In some embodiments, the passivation layer is between about 0.25 microns and 2 microns in thickness. In some embodiments, the passivation layer is between about 0.25 microns and 1 micron in thickness.

In some embodiments, the passivation layer is comprised of any suitable insulative low k dielectric material, including but not limited to silicon nitride or silicon dioxide. In some embodiments, the passivation layer is chosen from the group consisting of polyamids, carbon, doped silicon nitride, carbon doped silicon dioxide, fluorine doped silicon nitride, fluorine doped silicon dioxide, porous silicon dioxide, or any combinations thereof. In some embodiments, the passivation layer can comprise a dielectric ink capable of being screen-printed.

### Electrode Geometry

In some embodiments, the electrodes disclosed herein can be arranged in any manner suitable for practicing the methods disclosed herein.

In some embodiments, the electrodes are in a dot configuration, e.g. the electrodes comprises a generally circular or round configuration. In some embodiments, the angle of orientation between dots is from about 25° to about 60°. In some embodiments, the angle of orientation between dots is from about 30° to about 55°. In some embodiments, the angle of orientation between dots is from about 30° to about 50°. In some embodiments, the angle of orientation between dots is from about 35° to about 45°. In some embodiments, the angle of orientation between dots is about 25°. In some embodiments, the angle of orientation between dots is about 30°. In some embodiments, the angle of orientation between dots is about 35°. In some embodiments, the angle of orientation between dots is about 40°. In some embodiments, the angle of orientation between dots is about 45°. In some embodiments, the angle of orientation between dots is about 50°. In some embodiments, the angle of orientation between dots is about 55°. In some embodiments, the angle of orientation between dots is about 60°.

In some embodiments, the electrodes are in a substantially elongated configuration.

In some embodiments, the electrodes are in a configuration resembling wavy or nonlinear lines. In some embodiments, the array of electrodes is in a wavy or nonlinear line configuration, wherein the configuration comprises a repeating unit comprising the shape of a pair of dots connected by a linker, wherein the dots and linker define the boundaries of the electrode, wherein the linker tapers inward towards or at the midpoint between the pair of dots, wherein the diameters of the dots are the widest points along the length of the repeating unit, wherein the edge to edge distance between a parallel set of repeating units is equidistant, or roughly equidistant. In some embodiments, the electrodes are strips resembling wavy lines, as depicted in FIG. 8. In some embodiments, the edge to edge distance between the electrodes is equidistant, or roughly equidistant throughout the wavy line configuration. In some embodiments, the use of wavy line electrodes, as disclosed herein, lead to an enhanced DEP field gradient.

In some embodiments, the electrodes disclosed herein are in a planar configuration. In some embodiments, the electrodes disclosed herein are in a non-planar configuration.

In some embodiments, the devices disclosed herein surface selectively captures biomolecules on its surface. For example, the devices disclosed herein may capture biomolecules, such as nucleic acids, by, for example, a. nucleic acid hybridization; b. antibody—antigen interactions; c. biotin—avidin interactions; d. ionic or electrostatic interactions; or e. any combination thereof. The devices disclosed herein, therefore, may incorporate a functionalized surface which includes capture molecules, such as complementary nucleic acid probes, antibodies or other protein captures capable of capturing biomolecules (such as nucleic acids), biotin or other anchoring captures capable of capturing complementary target molecules such as avidin, capture molecules capable of capturing biomolecules (such as nucleic acids) by ionic or electrostatic interactions, or any combination thereof.

In some embodiments, the surface is functionalized to minimize and/or inhibit nonspecific binding interactions by: a. polymers (e.g., polyethylene glycol PEG); b. ionic or electrostatic interactions; c. surfactants; or d. any combination thereof. In some embodiments, the methods disclosed herein include use of additives which reduce non-specific binding interactions by interfering in such interactions, such as Tween 20 and the like, bovine serum albumin, nonspecific immunoglobulins, etc.

In some embodiments, the device comprises a plurality of microelectrode devices oriented (a) flat side by side, (b) facing vertically, or (c) facing horizontally. In other embodiments, the electrodes are in a sandwiched configuration, e.g. stacked on top of each other in a vertical format.

### Hydrogels

Overlaying electrode structures with one or more layers of materials can reduce the deleterious electrochemistry effects, including but not limited to electrolysis reactions, heating, and chaotic fluid movement that may occur on or near the electrodes, and still allow the effective separation of cells, bacteria, virus, nanoparticles, DNA, and other biomolecules to be carried out. In some embodiments, the materials layered over the electrode structures may be one or more porous layers. In other embodiments, the one or more porous layers is a polymer layer. In other embodiments, the one or more porous layers is a hydrogel.

In general, the hydrogel should have sufficient mechanical strength and be relatively chemically inert such that it will be able to endure the electrochemical effects at the electrode surface without disconfiguration or decomposition. In general, the hydrogel is sufficiently permeable to small aqueous ions, but keeps biomolecules away from the electrode surface.

In some embodiments, the hydrogel is a single layer, or coating.

In some embodiments, the hydrogel comprises a gradient of porosity, wherein the bottom of the hydrogel layer has greater porosity than the top of the hydrogel layer.

In some embodiments, the hydrogel comprises multiple layers or coatings. In some embodiments, the hydrogel comprises two coats. In some embodiments, the hydrogel comprises three coats. In some embodiments, the bottom (first) coating has greater porosity than subsequent coatings. In some embodiments, the top coat is has less porosity than the first coating. In some embodiments, the top coat has a mean pore diameter that functions as a size cut-off for particles of greater than 100 picometers in diameter.

In some embodiments, the hydrogel has a conductivity from about 0.001 S/m to about 10 S/m. In some embodiments, the hydrogel has a conductivity from about 0.01 S/m to about 10 S/m. In some embodiments, the hydrogel has a conductivity from about 0.1 S/m to about 10 S/m. In some embodiments, the hydrogel has a conductivity from about 1.0 S/m to about 10 S/m. In some embodiments, the hydrogel has a conductivity from about 0.01 S/m to about 5 S/m. In some embodiments, the hydrogel has a conductivity from about 0.01 S/m to about 4 S/m. In some embodiments, the hydrogel has a conductivity from about 0.01 S/m to about 3 S/m. In some embodiments, the hydrogel has a conductivity from about 0.01 S/m to about 2 S/m. In some embodiments, the hydrogel has a conductivity from about 0.1 S/m to about 5 S/m. In some embodiments, the hydrogel has a conductivity from about 0.1 S/m to about 4 S/m. In some embodiments, the hydrogel has a conductivity from about 0.1 S/m to about 3 S/m. In some embodiments, the hydrogel has a conductivity from about 0.1 S/m to about 2 S/m. In some embodiments, the hydrogel has a conductivity from about 0.1 S/m to about 1.5 S/m. In some embodiments, the hydrogel has a conductivity from about 0.1 S/m to about 1.0 S/m.

In some embodiments, the hydrogel has a conductivity of about 0.1 S/m. In some embodiments, the hydrogel has a conductivity of about 0.2 S/m. In some embodiments, the hydrogel has a conductivity of about 0.3 S/m. In some embodiments, the hydrogel has a conductivity of about 0.4 S/m. In some embodiments, the hydrogel has a conductivity of about 0.5 S/m. In some embodiments, the hydrogel has a conductivity of about 0.6 S/m. In some embodiments, the hydrogel has a conductivity of about 0.7 S/m. In some embodiments, the hydrogel has a conductivity of about 0.8 S/m. In some embodiments, the hydrogel has a conductivity of about 0.9 S/m. In some embodiments, the hydrogel has a conductivity of about 1.0 S/m.

In some embodiments, the hydrogel has a thickness from about 0.1 microns to about 10 microns. In some embodiments, the hydrogel has a thickness from about 0.1 microns to about 5 microns. In some embodiments, the hydrogel has a thickness from about 0.1 microns to about 4 microns. In some embodiments, the hydrogel has a thickness from about 0.1 microns to about 3 microns. In some embodiments, the hydrogel has a thickness from about 0.1 microns to about 2 microns. In some embodiments, the hydrogel has a thickness from about 1 micron to about 5 microns. In some embodiments, the hydrogel has a thickness from about 1 micron to about 4 microns. In some embodiments, the hydrogel has a thickness from about 1 micron to about 3 microns. In some embodiments, the hydrogel has a thickness from about 1 micron to about 2 microns. In some embodiments, the hydrogel has a thickness from about 0.5 microns to about 1 micron.

In some embodiments, the viscosity of a hydrogel solution prior to spin-coating ranges from about 0.5 cP to about 5 cP. In some embodiments, a single coating of hydrogel solution has a viscosity of between about 0.75 cP and 5 cP prior to spin-coating. In some embodiments, in a multi-coat hydrogel, the first hydrogel solution has a viscosity from about 0.5 cP to about 1.5 cP prior to spin coating. In some embodiments, the second hydrogel solution has a viscosity from about 1 cP to about 3 cP. The viscosity of the hydrogel solution is based on the polymers concentration (0.1%-10%) and polymers molecular weight (10,000 to 300,000) in the solvent and the starting viscosity of the solvent.

In some embodiments, the first hydrogel coating has a thickness between about 0.5 microns and 1 micron. In some embodiments, the first hydrogel coating has a thickness between about 0.5 microns and 0.75 microns. In some embodioments, the first hydrogel coating has a thickness between about 0.75 and 1 micron. In some embodiments, the second hydrogel coating has a thickness between about 0.2 microns and 0.5 microns. In some embodiments, the second hydrogel coating has a thickness between about 0.2 and 0.4 microns. In some embodiments, the second hydrogel coating has a thickness between about 0.2 and 0.3 microns. In some embodiments, the second hydrogel coating has a thickness between about 0.3 and 0.4 microns.

In some embodiments, the hydrogel comprises any suitable synthetic polymer forming a hydrogel. In general, any sufficiently hydrophilic and polymerizable molecule may be utilized in the production of a synthetic polymer hydrogel for use as disclosed herein. Polymerizable moieties in the monomers may include alkenyl moieties including but not limited to substituted or unsubstituted a,f3,unsaturated carbonyls wherein the double bond is directly attached to a carbon which is double bonded to an oxygen and single bonded to another oxygen, nitrogen, sulfur, halogen, or carbon; vinyl, wherein the double bond is singly bonded to an oxygen, nitrogen, halogen, phosphorus or sulfur; allyl, wherein the double bond is singly bonded to a carbon which is bonded to an oxygen, nitrogen, halogen, phosphorus or sulfur; homoallyl, wherein the double bond is singly bonded to a carbon which is singly bonded to another carbon which is then singly bonded to an oxygen, nitrogen, halogen, phosphorus or sulfur; alkynyl moieties wherein a triple bond exists between two carbon atoms. In some embodiments, acryloyl or acrylamido monomers such as acrylates, methacrylates, acrylamides, methacrylamides, etc., are useful for formation of hydrogels as disclosed herein. More preferred acrylamido monomers include acrylamides, N-substituted acrylamides, N-substituted methacrylamides, and methacrylamide. In some embodiments, a hydrogel comprises polymers such as epoxide-based polymers, vinyl-based polymers, allyl-based polymers, homoallyl-based polymers, cyclic anhydride-based polymers, ester-based polymers, ether-based polymers, alkylene-glycol based polymers (e.g., polypropylene glycol), and the like.

In some embodiments, the hydrogel comprises polyhydroxyethylmethacrylate (pHEMA), cellulose acetate, cellulose acetate phthalate, cellulose acetate butyrate, or any appropriate acrylamide or vinyl-based polymer, or a derivative thereof.

In some embodiments, the hydrogel is applied by vapor deposition.

In some embodiments, the hydrogel is polymerized via atom-transfer radical-polymerization via (ATRP).

In some embodiments, the hydrogel is polymerized via reversible addition-fragmentation chain-transfer (RAFT) polymerization.

In some embodiments, additives are added to a hydrogel to increase conductivity of the gel. In some embodiments, hydrogel additives are conductive polymers (e.g., PEDOT: PSS), salts (e.g., copper chloride), metals (e.g., gold), plasticizers (e.g., PEG200, PEG 400, or PEG 600), or co-solvents.

In some embodiments, the hydrogel also comprises compounds or materials which help maintain the stability of the DNA hybrids, including, but not limited to histidine, histidine peptides, polyhistidine, lysine, lysine peptides, and other cationic compounds or substances. Dielectrophoretic Fields

In some embodiments, the methods, devices and systems described herein provide a mechanism to collect, separate, or isolate cells, particles, and/or molecules (such as nucleic acid) from a fluid material (which optionally contains other materials, such as contaminants, residual cellular material, or the like).

In some embodiments, an AC electrokinetic field is generated to collect, separate or isolate biomolecules, such as nucleic acids. In some embodiments, the AC electrokinetic field is a dielectrophoretic field. Accordingly, in some embodiments dielectrophoresis (DEP) is utilized in various steps of the methods described herein.

In some embodiments, the devices and systems described herein are capable of generating DEP fields, and the like. In specific embodiments, DEP is used to concentrate cells and/or nucleic acids (e.g., concurrently or at different times). In certain embodiments, methods described herein further comprise energizing the array of electrodes so as to produce the first, second, and any further optional DEP fields. In some embodiments, the devices and systems described herein are capable of being energized so as to produce the first, second, and any further optional DEP fields.

DEP is a phenomenon in which a force is exerted on a dielectric particle when it is subjected to a non-uniform electric field. Depending on the step of the methods described herein, aspects of the devices and systems described herein, and the like, the dielectric particle in various embodiments herein is a biological cell and/or a molecule, such as a nucleic acid molecule. Different steps of the methods described herein or aspects of the devices or systems described herein may be utilized to isolate and separate different components, such as intact cells or other particular material; further, different field regions of the DEP field may be used in different steps of the methods or aspects of the devices and systems described herein. This dielectrophoretic force does not require the particle to be charged. In some instances, the strength of the force depends on the medium and the specific particles' electrical properties, on the particles' shape and size, as well as on the frequency of the electric field. In some instances, fields of a particular frequency selectivity manipulate particles. In certain aspects described herein, these processes allow for the separation of cells and/or smaller particles (such as molecules, including nucleic acid molecules) from other components (e.g., in a fluid medium) or each other.

In various embodiments provided herein, a method described herein comprises producing a first DEP field region and a second DEP field region with the array. In various embodiments provided herein, a device or system described herein is capable of producing a first DEP field region and a second DEP field region with the array. In some instances, the first and second field regions are part of a single field (e.g., the first and second regions are present at the same time, but are found at different locations within the device and/or upon the array). In some embodiments, the first and second field regions are different fields (e.g. the first region is created by energizing the electrodes at a first time, and the second region is created by energizing the electrodes a second time). In specific aspects, the first DEP field region is suitable for concentrating or isolating cells (e.g., into a low field DEP region). In some embodiments, the second DEP field region is suitable for concentrating smaller particles, such as molecules (e.g., nucleic acid), for example into a high field DEP region. In some instances, a method described herein optionally excludes use of either the first or second DEP field region.

In some embodiments, the first DEP field region is in the same chamber of a device as disclosed herein as the second DEP field region. In some embodiments, the first DEP field region and the second DEP field region occupy the same area of the array of electrodes.

In some embodiments, the first DEP field region is in a separate chamber of a device as disclosed herein, or a separate device entirely, from the second DEP field region.

### DEP Field Region

In some aspects, e.g., high conductance buffers (>100 mS/m), the method described herein comprises applying a fluid comprising cell-free DNA to a device comprising an array of electrodes, and, thereby, concentrating the cell-free DNA in a first DEP field region. In some embodiments, a first DEP field region will concentrate cell-free DNA fragments that are a first size or size range and a second DEP field region will concentrate cell-free DNA fragments that are a second size or size range. Subsequent or concurrent optional third and fourth DEP regions, may collect or isolate DNA fragments at yet more sizes or size ranges or may isolate other fluid components, including biomolecules, such as proteins or RNA.

The first DEP field region may be any field region suitable for concentrating cells from a fluid. For this application, the cell-free DNA fragments are generally concentrated near the array of electrodes. In some embodiments, the first DEP field region is a dielectrophoretic low field region. In some embodiments, the first DEP field region is a dielectrophoretic high field region. In some aspects, e.g. low conductance buffers (<100 mS/m), the method described herein comprises applying a fluid comprising cell-free DNA fragments to a device comprising an array of electrodes, and, thereby, concentrating the cell-free DNA fragments or other particulate material in a first DEP field region.

High versus low field capture is generally dependent on the conductivity of the fluid, wherein generally, the crossover point is between about 300-500 mS/m. In some embodiments, the first DEP field region is a dielectrophoretic low field region performed in fluid conductivity of greater than about 300 mS/m. In some embodiments, the first DEP field region is a dielectrophoretic low field region performed in fluid conductivity of less than about 300 mS/m. In some embodiments, the first DEP field region is a dielectrophoretic high field region performed in fluid conductivity of greater than about 300 mS/m. In some embodiments, the first DEP field region is a dielectrophoretic high field region performed in fluid conductivity of less than about 300 mS/m. In some embodiments, the first DEP field region is a dielectrophoretic low field region performed in fluid conductivity of greater than about 500 mS/m. In some embodiments, the first DEP field region is a dielectrophoretic low field region performed in fluid conductivity of less than about 500 mS/m. In some embodiments, the first DEP field region is a dielectrophoretic high field region performed in fluid conductivity of greater than about 500 mS/m. In some embodiments, the first DEP field region is a dielectrophoretic high field region performed in fluid conductivity of less than about 500 mS/m.

In some embodiments, the first dielectrophoretic field region is produced by an alternating current. The alternating current has any amperage, voltage, frequency, and the like suitable for concentrating cell-free DNA fragments. In some embodiments, the first dielectrophoretic field region is produced using an alternating current having an amperage of 0.1 micro Amperes-10 Amperes; a voltage of 1-50 Volts peak to peak; and/or a frequency of 1-10,000,000 Hz. In some embodiments, the first DEP field region is produced using an alternating current having a voltage of 5-25 volts peak to peak. In some embodiments, the first DEP field region is produced using an alternating current having a frequency of from 3-15 kHz. In some embodiments, the first DEP field region is produced using an alternating current having an amperage of 1 milliamp to 1 amp. In some embodiments, the first DEP field region is produced using an alternating current having an amperage of 0.1 micro Amperes-1 Ampere. In some embodiments, the first DEP field region is produced using an alternating current having an amperage of 1 micro Amperes-1 Ampere. In some embodiments, the first DEP field region is produced using an alternating current having an amperage of 100 micro Amperes-1 Ampere. In some embodiments, the first DEP field region is produced using an alternating current having an amperage of 500 micro Amperes-500 milli Amperes. In some embodiments, the first DEP field region is produced using an alternating current having a voltage of 1-25 Volts peak to peak. In some embodiments, the first DEP field region is produced using an alternating current having a voltage of 1-10 Volts peak to peak. In some embodiments, the first DEP field region is produced using an alternating current having a voltage of 25-50 Volts peak to peak. In some embodiments, the first DEP field region is produced using a frequency of from 10-1,000,000 Hz. In some embodiments, the first DEP field region is produced using a frequency of from 100-100,000 Hz. In some embodiments, the first DEP field region is produced using a frequency of from 100-10,000 Hz. In some embodiments, the first DEP field region is produced using a frequency of from 10,000-100,000 Hz. In some embodiments, the first DEP field region is produced using a frequency of from 100,000-1,000,000 Hz.

In some embodiments, the first dielectrophoretic field region is produced by a direct current. The direct current has any amperage, voltage, frequency, and the like suitable for concentrating cells. In some embodiments, the first dielectrophoretic field region is produced using a direct current having an amperage of 0.1micro Amperes-1 Amperes; a voltage of 10 milli Volts-10 Volts; and/or a pulse width of 1 milliseconds-1000 seconds and a pulse frequency of 0.001-1000 Hz. In some embodiments, the first DEP field region is produced using a direct current having an amperage of 1 micro Amperes-1 Amperes. In some embodiments, the first DEP field region is produced using a direct current having an amperage of 100 micro Amperes-500 milli Amperes. In some embodiments, the first DEP field region is produced using a direct current having an amperage of 1 milli Amperes-1 Amperes. In some embodiments, the first DEP field region is produced using a direct current having an amperage of 1 micro Amperes-1 milli Amperes. In some embodiments, the first DEP field region is produced using a direct current having a pulse width of 500 milliseconds-500 seconds. In some embodiments, the first DEP field region is produced using a direct current having a pulse width of 500 milliseconds-100 seconds. In some embodiments, the first DEP field region is produced using a direct current having a pulse width of 1 second-1000 seconds. In some embodiments, the first DEP field region is produced using a direct current having a pulse width of 500 milliseconds-1 second. In some embodiments, the first DEP field region is produced using a pulse frequency of 0.01-1000 Hz. In some embodiments, the first DEP field region is produced using a pulse frequency of 0.1-100 Hz. In some embodiments, the first DEP field region is produced using a pulse frequency of 1-100 Hz. In some embodiments, the first DEP field region is produced using a pulse frequency of 100-1000 Hz.

In some embodiments, the fluid also comprises cells. In some embodiments, the fluid comprises a mixture of cell types. For example, blood comprises red blood cells and white blood cells. In some embodiments, one cell type (or any number of cell types less than the total number of cell types comprising the sample) is preferentially concentrated in a different DEP field than the DEP field in which the cell-free DNA is concentrated.

In some embodiments, a method, device or system described herein is suitable for isolating or separating cell-free DNA fragments of specific sizes. In some embodiments, the DEP field of the method, device or system is specifically tuned to allow for the separation or concentration of a specific size of cell-free DNA fragment into a field region of the DEP field. In some embodiments, a method, device or system described herein provides more than one field region wherein more than one size of cell-free DNA is preferentially isolated or concentrated. In some embodiments, a method, device, or system described herein is tunable so as to allow isolation or concentration of different sizes of cell-free DNA fragments within the DEP field regions thereof. In some embodiments, a method provided herein further comprises tuning the DEP field. In some embodiments, a device or system provided herein is capable of having the DEP field tuned. In some instances, such tuning may be in providing a DEP particularly suited for the desired purpose. For example, modifications in the array, the energy, or another parameter are optionally utilized to tune the DEP field. Tuning parameters for finer resolution include electrode diameter, edge to edge distance between electrodes, voltage, frequency, fluid conductivity and hydrogel composition.

In some embodiments, the first DEP field region comprises the entirety of an array of electrodes. In some embodiments, the first DEP field region comprises a portion of an array of electrodes. In some embodiments, the first DEP field region comprises about 90%, about 80%, about 70%, about 60%, about 50%, about 40%, about 30%, about 25%, about 20%, or about 10% of an array of electrodes. In some embodiments, the first DEP field region comprises about a third of an array of electrodes.

### Isolating Nucleic Acids

In one aspect, described herein is a method for isolating a nucleic acid from a fluid. In some embodiments, the nucleic acids are cell-free nucleic acids. In some embodiments, disclosed herein is method for isolating a cell-free nucleic acid from a fluid, the method comprising: a. applying the fluid to a device, the device comprising an array of electrodes; b. concentrating a plurality of cellular materials in a first AC electrokinetic (e.g., dielectrophoretic) field region; c. isolating nucleic acid in a second AC electrokinetic (e.g., dielectrophoretic) field region; and d. flushing the cellular materials away. In some instances, residual cellular material is concentrated near the low field region. In some embodiments, the residual material is washed from the device and/or washed from the nucleic acids. In some embodiments, the nucleic acid is concentrated in the second AC electrokinetic field region.

In one aspect, described herein is a method for isolating a nucleic acid from a fluid comprising cells or other particulate material. In some embodiments, the nucleic acids are not inside the cells (e.g., cell-free DNA in fluid). In some embodiments, disclosed herein is a method for isolating a nucleic acid from a fluid comprising cells or other particulate material, the method comprising: a. applying the fluid to a device, the device comprising an array of electrodes; b. concentrating a plurality of cells in a first AC electrokinetic (e.g., dielectrophoretic) field region; c. isolating nucleic acid in a second AC electrokinetic (e.g., dielectrophoretic) field region; and d. flushing cells away. In some embodiments, the method further comprises degrading residual proteins after flushing cells away. In some embodiments, cells are concentrated on or near a low field region and nucleic acids are concentrated on or near a high field region. In some instances, the cells are washed from the device and/or washed from the nucleic acids.

In one aspect, the methods, systems and devices described herein isolate nucleic acid from a fluid. In some embodiments, the fluid is a liquid, optionally water or an aqueous solution or dispersion. In some embodiments, the fluid is any suitable fluid including a bodily fluid. Exemplary bodily fluids include blood, serum, plasma, bile, milk, cerebrospinal fluid, gastric juice, ejaculate, mucus, peritoneal fluid, saliva, sweat, tears, urine, and the like. In some embodiments, nucleic acids are isolated from bodily fluids using the methods, systems or devices described herein as part of a medical therapeutic or diagnostic procedure, device or system. In some embodiments, the fluid is tissues and/or cells solubilized and/or dispersed in a fluid. For example, the tissue can be a cancerous tumor from which nucleic acid can be isolated using the methods, devices or systems described herein.

In some embodiments, the fluid is water.

In some embodiments, the fluid may also comprise other particulate material. Such particulate material may be, for example, inclusion bodies (e.g., ceroids or Mallory bodies), cellular casts (e.g., granular casts, hyaline casts, cellular casts, waxy casts and pseudo casts), Pick's bodies, Lewy bodies, fibrillary tangles, fibril formations, cellular debris and other particulate material. In some embodiments, particulate material is an aggregated protein (e.g., beta-amyloid).

The fluid can have any conductivity including a high or low conductivity. In some embodiments, the conductivity is between about 1 μS/m to about 10 mS/m. In some embodiments, the conductivity is between about 10 μS/m to about 10 mS/m. In other embodiments, the conductivity is between about 50 μS/m to about 10 mS/m. In yet other embodiments, the conductivity is between about 100 μS/m to about 10 mS/m, between about 100 μS/m to about 8 mS/m, between about 100 μS/m to about 6 mS/m, between about 100 μS/m to about 5 mS/m, between about 100 μS/m to about 4 mS/m, between about 100 μS/m to about 3 mS/m, between about 100 μS/m to about 2 mS/m, or between about 100 μS/m to about 1 mS/m.

In some embodiments, the conductivity is about 1 μS/m. In some embodiments, the conductivity is about 10 μS/m. In some embodiments, the conductivity is about 100 μS/m. In some embodiments, the conductivity is about 1 mS/m. In other embodiments, the conductivity is about 2 mS/m. In some embodiments, the conductivity is about 3 mS/m. In yet other embodiments, the conductivity is about 4 mS/m. In some embodiments, the conductivity is about 5 mS/m. In some embodiments, the conductivity is about 10 mS/m. In still other embodiments, the conductivity is about 100 mS/m. In some embodiments, the conductivity is about 1 S/m. In other embodiments, the conductivity is about 10 S/m.

In some embodiments, the conductivity is at least 1 μS/m. In yet other embodiments, the conductivity is at least 10 μS/m. In some embodiments, the conductivity is at least 100 μS/m. In some embodiments, the conductivity is at least 1 mS/m. In additional embodiments, the conductivity is at least 10 mS/m. In yet other embodiments, the conductivity is at least 100 mS/m. In some embodiments, the conductivity is at least 1 S/m. In some embodiments, the conductivity is at least 10 S/m. In some embodiments, the conductivity is at most 1 μS/m. In some embodiments, the conductivity is at most 10 μS/m. In other embodiments, the conductivity is at most 100 μS/m. In some embodiments, the conductivity is at most 1 mS/m. In some embodiments, the conductivity is at most 10 mS/m. In some embodiments, the conductivity is at most 100 mS/m. In yet other embodiments, the conductivity is at most 1 S/m. In some embodiments, the conductivity is at most 10 S/m.

In some embodiments, the fluid is a small volume of liquid including less than 10 ml. In some embodiments, the fluid is less than 8 ml. In some embodiments, the fluid is less than 5 ml. In some embodiments, the fluid is less than 2 ml. In some embodiments, the fluid is less than 1 ml. In some embodiments, the fluid is less than 500 μl. In some embodiments, the fluid is less than 200 μl. In some embodiments, the fluid is less than 100 μl. In some embodiments, the fluid is less than 50 μl. In some embodiments, the fluid is less than 10 μl. In some embodiments, the fluid is less than 5 μl. In some embodiments, the fluid is less than 1 μl.

In some embodiments, the quantity of fluid applied to the device or used in the method comprises less than about 100,000,000 cells. In some embodiments, the fluid comprises less than about 10,000,000 cells. In some embodiments, the fluid comprises less than about 1,000,000 cells. In some embodiments, the fluid comprises less than about 100,000 cells. In some embodiments, the fluid comprises less than about 10,000 cells. In some embodiments, the fluid comprises less than about 1,000 cells. In some embodiments, the fluid is cell-free.

In some embodiments, isolation of nucleic acid from a fluid comprising cells or other particulate material with the devices, systems and methods described herein takes less than about 30 minutes, less than about 20 minutes, less than about 15 minutes, less than about 10 minutes, less than about 5 minutes or less than about 1 minute. In other embodiments, isolation of nucleic acid from a fluid comprising cells or other particulate material with the devices, systems and methods described herein takes not more than 30 minutes, not more than about 20 minutes, not more than about 15 minutes, not more than about 10 minutes, not more than about 5 minutes, not more than about 2 minutes or not more than about 1 minute. In additional embodiments, isolation of nucleic acid from a fluid comprising cells or other particulate material with the devices, systems and methods described herein takes less than about 15 minutes, preferably less than about 10 minutes or less than about 5 minutes.

In some instances, extra-cellular DNA, cell-free DNA fragments, or other nucleic acids (outside cells) are isolated from a fluid comprising cells of other particulate material. In some embodiments, the fluid comprises cells. In some embodiments, the fluid does not comprise cells.

### Removal of Residual Material

In some embodiments, following concentration of the targeted cellular material in the second DEP field region, the method includes optionally flushing residual material from the targeted cellular material. In some embodiments, the devices or systems described herein are capable of optionally comprising a reservoir comprising a fluid suitable for flushing residual material from the targeted cellular material. In some embodiments, the targeted cellular material is held near the array of electrodes, such as in the second DEP field region, by continuing to energize the electrodes. “Residual material” is anything originally present in the fluid, originally present in the cells, added during the procedure, created through any step of the process including but not limited to lysis of the cells (i.e. residual cellular material), and the like. For example, residual material includes non-lysed cells, cell wall fragments, proteins, lipids, carbohydrates, minerals, salts, buffers, plasma, and undesired nucleic acids. In some embodiments, the lysed cellular material comprises residual protein freed from the plurality of cells upon lysis. It is possible that not all of the targeted cellular material will be concentrated in the second DEP field. In some embodiments, a certain amount of targeted cellular material is flushed with the residual material.

In some embodiments, the residual material is flushed in any suitable fluid, for example in water, TBE buffer, or the like. In some embodiments, the residual material is flushed with any suitable volume of fluid, flushed for any suitable period of time, flushed with more than one fluid, or any other variation. In some embodiments, the method of flushing residual material is related to the desired level of isolation of the targeted cellular material with higher purity targeted cellular material requiring more stringent flushing and/or washing. In other embodiments, the method of flushing residual material is related to the particular starting material and its composition. In some instances, a starting material that is high in lipid requires a flushing procedure that involves a hydrophobic fluid suitable for solubilizing lipids.

In some embodiments, the method includes degrading residual material including residual protein. In some embodiments, the devices or systems are capable of degrading residual material including residual protein. For example, proteins are degraded by one or more of chemical degradation (e.g. acid hydrolysis) and enzymatic degradation. In some embodiments, the enzymatic degradation agent is a protease. In other embodiments, the protein degradation agent is Proteinase K. The optional step of degradation of residual material is performed for any suitable time, temperature, and the like. In some embodiments, the degraded residual material (including degraded proteins) is flushed from the nucleic acid.

In some embodiments, the agent used to degrade the residual material is inactivated or degraded. In some embodiments, the devices or systems are capable of degrading or inactivating the agent used to degrade the residual material. In some embodiments, an enzyme used to degrade the residual material is inactivated by heat (e.g., 50 to 95° C. for 5-15 minutes). For example, enzymes including proteases, (for example, Proteinase K) are degraded and/or inactivated using heat (typically, 15 minutes, 70° C.). In some embodiments wherein the residual proteins are degraded by an enzyme, the method further comprises inactivating the degrading enzyme (e.g., Proteinase K) following degradation of the proteins. In some embodiments, heat is provided by a heating module in the device (temperature range, e.g., from 30 to 95° C.).

The order and/or combination of certain steps of the method can be varied. In some embodiments, the devices or methods are capable of performing certain steps in any order or combination. For example, in some embodiments, the residual material and the degraded proteins are flushed in separate or concurrent steps. That is, the residual material is flushed, followed by degradation of residual proteins, followed by flushing degraded proteins from the nucleic acid. In some embodiments, one first degrades the residual proteins, and then flush both the residual material and degraded proteins from the nucleic acid in a combined step.

In some embodiments, the targeted cellular materials are retained in the device and optionally used in further procedures such as PCR or other procedures manipulating or amplifying nucleic acid. In some embodiments, the devices and systems are capable of performing PCR or other optional procedures. In other embodiments, the targeted cellular materials are collected and/or eluted from the device. In some embodiments, the devices and systems are capable of allowing collection and/or elution of targeted cellular material from the device or system. In some embodiments, the isolated cellular material is collected by (i) turning off the second dielectrophoretic field region; and (ii) eluting the material from the array in an eluant. Exemplary eluants include water, TE, TBE and L-Histidine buffer.

### Nucleic Acids and Yields Thereof

In some embodiments, the method, device, or system described herein is optionally utilized to obtain, isolate, or separate any desired nucleic acid that may be obtained from such a method, device or system. Nucleic acids isolated by the methods, devices and systems described herein include DNA (deoxyribonucleic acid), RNA (ribonucleic acid), and combinations thereof. DNA can include cell-free DNA and DNA fragments. In some embodiments, the nucleic acid is isolated in a form suitable for sequencing or further manipulation of the nucleic acid, including amplification, ligation or cloning.

In some embodiments, the isolated, separated, or captured nucleic acid comprises DNA fragments that are selectively or preferentially isolated, separated, or captured based on their sizes. In some embodiments, the DNA fragments that are selectively or preferentially isolated, separated, or captured are between 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, and/or 300-500 bp in length. In some embodiments, the DNA fragments that are selectively or preferentially isolated, separated, or captured are between 600-700 bp, 700-800 bp, 800-900 bp, 900-1000 bp, 1-2 kbp, 2-3 kbp, 3-4 kbp, 4-5 kbp, 5-6 kbp, 6-7 kbp, 7-8 kbp, 8-9 kbp, or 9-10 kbp. In some embodiments, the DNA fragments that are selectively or preferentially isolated, separated, or captured are greater than 300, 400, 500, 600, 700, 800, 900, or 1000 bp in size.

In some embodiments, the DNA fragments are cell-free DNA fragments.

In various embodiments, an isolated or separated nucleic acid is a composition comprising nucleic acid that is free from at least 99% by mass of other materials, free from at least 99% by mass of residual cellular material (e.g., from lysed cells from which the nucleic acid is obtained), free from at least 98% by mass of other materials, free from at least 98% by mass of residual cellular material, free from at least 95% by mass of other materials, free from at least 95% by mass of residual cellular material, free from at least 90% by mass of other materials, free from at least 90% by mass of residual cellular material, free from at least 80% by mass of other materials, free from at least 80% by mass of residual cellular material, free from at least 70% by mass of other materials, free from at least 70% by mass of residual cellular material, free from at least 60% by mass of other materials, free from at least 60% by mass of residual cellular material, free from at least 50% by mass of other materials, free from at least 50% by mass of residual cellular material, free from at least 30% by mass of other materials, free from at least 30% by mass of residual cellular material, free from at least 10% by mass of other materials, free from at least 10% by mass of residual cellular material, free from at least 5% by mass of other materials, or free from at least 5% by mass of residual cellular material.

In various embodiments, the nucleic acid has any suitable purity. For example, if a DNA sequencing procedure can work with nucleic acid samples having about 20% residual cellular material, then isolation of the nucleic acid to 80% is suitable. In some embodiments, the isolated nucleic acid comprises less than about 80%, less than about 70%, less than about 60%, less than about 50%, less than about 40%, less than about 30%, less than about 20%, less than about 10%, less than about 5%, or less than about 2% non-nucleic acid cellular material and/or protein by mass. In some embodiments, the isolated nucleic acid comprises greater than about 99%, greater than about 98%, greater than about 95%, greater than about 90%, greater than about 80%, greater than about 70%, greater than about 60%, greater than about 50%, greater than about 40%, greater than about 30%, greater than about 20%, or greater than about 10% nucleic acid by mass.

The nucleic acids are isolated in any suitable form including unmodified, derivatized, fragmented, non-fragmented, and the like. In some embodiments, the nucleic acid is collected in a form suitable for sequencing. In some embodiments, the nucleic acid is collected in a fragmented form suitable for shotgun-sequencing, amplification or other manipulation. The nucleic acid may be collected from the device in a solution comprising reagents used in, for example, a DNA sequencing procedure, such as nucleotides as used in sequencing by synthesis methods.

In some embodiments, the methods described herein result in an isolated nucleic acid sample that is approximately representative of the nucleic acid of the starting sample. In some embodiments, the devices and systems described herein are capable of isolating nucleic acid from a sample that is approximately representative of the nucleic acid of the starting sample. That is, the population of nucleic acids collected by the method, or capable of being collected by the device or system, are substantially in proportion to the population of nucleic acids present in the cells in the fluid. In some embodiments, this aspect is advantageous in applications in which the fluid is a complex mixture of many cell types and the practitioner desires a nucleic acid-based procedure for determining the relative populations of the various cell types.

In some embodiments, the nucleic acid isolated using the methods described herein or capable of being isolated by the devices described herein is high-quality and/or suitable for using directly in downstream procedures such as DNA sequencing, nucleic acid amplification, such as PCR, or other nucleic acid manipulation, such as ligation, cloning or further translation or transformation assays. In some embodiments, the collected nucleic acid comprises at most 0.01% protein. In some embodiments, the collected nucleic acid comprises at most 0.5% protein. In some embodiments, the collected nucleic acid comprises at most 0.1% protein. In some embodiments, the collected nucleic acid comprises at most 1% protein. In some embodiments, the collected nucleic acid comprises at most 2% protein. In some embodiments, the collected nucleic acid comprises at most 3% protein. In some embodiments, the collected nucleic acid comprises at most 4% protein. In some embodiments, the collected nucleic acid comprises at most 5% protein.

In some embodiments, the nucleic acid isolated by the methods described herein or capable of being isolated by the devices described herein has a concentration of at least 0.5 ng/mL. In some embodiments, the nucleic acid isolated by the methods described herein or capable of being isolated by the devices described herein has a concentration of at least 1 ng/mL. In some embodiments, the nucleic acid isolated by the methods described herein or capable of being isolated by the devices described herein has a concentration of at least 5 ng/mL. In some embodiments, the nucleic acid isolated by the methods described herein or capable of being isolated by the devices described herein has a concentration of at least 10 ng/ml.

In some embodiments, about 50 pico-grams of nucleic acid is isolated from about 5,000 cells using the methods, systems or devices described herein. In some embodiments, the methods, systems or devices described herein yield at least 10 pico-grams of nucleic acid from about 5,000 cells. In some embodiments, the methods, systems or devices described herein yield at least 20 pico-grams of nucleic acid from about 5,000 cells. In some embodiments, the methods, systems or devices described herein yield at least 50 pico-grams of nucleic acid from about 5,000 cells. In some embodiments, the methods, systems or devices described herein yield at least 75 pico-grams of nucleic acid from about 5,000 cells. In some embodiments, the methods, systems or devices described herein yield at least 100 pico-grams of nucleic acid from about 5,000 cells. In some embodiments, the methods, systems or devices described herein yield at least 200 pico-grams of nucleic acid from about 5,000 cells. In some embodiments, the methods, systems or devices described herein yield at least 300 pico-grams of nucleic acid from about 5,000 cells. In some embodiments, the methods, systems or devices described herein yield at least 400 pico-grams of nucleic acid from about 5,000 cells.In some embodiments, the methods, systems or devices described herein yield at least 500 pico-grams of nucleic acid from about 5,000 cells. In some embodiments, the methods, systems or devices described herein yield at least 1,000 pico-grams of nucleic acid from about 5,000 cells. In some embodiments, the methods, systems or devices described herein yield at least 10,000 pico-grams of nucleic acid from about 5,000 cells.

### Assays and Applications

In some embodiments, the amount of nucleic acids, including the amounts of nucleic acids of particular sizes, can be quantified. In some embodiments, the nucleic acids can be quantified by making them visualizable, including, for example, by staining the nucleic acids with a label, dye or otherwise tagging the nucleic acids for quantification, identification and/or tracing. The methods and devices disclosed herein may employ dyes, including intercalating dyes, antibody labeling, stains and other imaging molecules that enable direct quantification of the cell-free biomarker materials directly on or in use with the embodied devices, including the use of fluorescence microscopy. Examples of fluorescent labeling of particulates, e.g. DNA and RNA, include but are not limited to cyanine dimers high-affinity stains (Life Technologies) can used. Examples include YOYO®-1, YOYO®-3, POPO™-1, POPO™-3, TOTO®-1, and TOTO®-3, SYBR Green I, SYBR Green II, SYBR Gold stains, SYBR DX, SYTO 10, SYTO17, SYTO-13, SYBR14, SYTO-82, and ethidium bromide.

In some embodiments, the methods described herein further comprise optionally amplifying the isolated nucleic acid by polymerase chain reaction (PCR). In some embodiments, the PCR reaction is performed on or near the array of electrodes or in the device. In some embodiments, the device or system comprise a heater and/or temperature control mechanisms suitable for thermocycling.

PCR is optionally done using traditional thermocycling by placing the reaction chemistry analytes in between two efficient thermoconductive elements (e.g., aluminum or silver) and regulating the reaction temperatures using TECs. Additional designs optionally use infrared heating through optically transparent material like glass or thermo polymers. In some instances, designs use smart polymers or smart glass that comprise conductive wiring networked through the substrate. This conductive wiring enables rapid thermal conductivity of the materials and (by applying appropriate DC voltage) provides the required temperature changes and gradients to sustain efficient PCR reactions. In certain instances, heating is applied using resistive chip heaters and other resistive elements that will change temperature rapidly and proportionally to the amount of current passing through them.

The methods and devices disclosed herein may also use Quantitative Real Time PCR, including of nuclear or mitochondrial DNA or other target nucleic acid molecule markers, enzyme-linked immunosorbent assays (ELISA), direct SYBR gold assays, direct PicoGreen assays, loss of heterozygosity (LOH) of microsatellite markers, optionally followed by electrophoresis analysis, including but not limited to capillary electrophoresis analysis, sequencing and/or cloning, including next generation sequencing, methylation analysis, including but not limited to modified semi-nested or nested methylation specific PCR, DNA specific PCR (MSP), quantification of minute amounts of DNA after bisulfitome amplification (qMAMBRA), as well as methylation on beads, mass-based analysis, including but not limited to MALDI-ToF (matrix-assisted laser desorption/ionization time of flight analysis, optionally in combination with PCR, and digital PCR.

In some embodiments, used in conjunction with traditional fluorometry (ccd, pmt, other optical detector, and optical filters), fold amplification is monitored in real-time or on a timed interval. In certain instances, quantification of final fold amplification is reported via optical detection converted to AFU (arbitrary fluorescence units correlated to analyze doubling) or translated to electrical signal via impedance measurement or other electrochemical sensing.

Given the small size of the micro electrode array, these elements are optionally added around the micro electrode array and the PCR reaction will be performed in the main sample processing chamber (over the DEP array) or the analytes to be amplified are optionally transported via fluidics to another chamber within the fluidic cartridge to enable on-cartridge Lab-On-Chip processing.

In some instances, light delivery schemes are utilized to provide the optical excitation and/or emission and/or detection of fold amplification. In certain embodiments, this includes using the flow cell materials (thermal polymers like acrylic (PMMA) cyclic olefin polymer (COP), cyclic olefin co-polymer, (COC), etc. . . ) as optical wave guides to remove the need to use external components. In addition, in some instances light sources—light emitting diodes—LEDs, vertical-cavity surface-emitting lasers—VCSELs, and other lighting schemes are integrated directly inside the flow cell or built directly onto the micro electrode array surface to have internally controlled and powered light sources. Miniature PMTs, CCDs, or CMOS detectors can also be built into the flow cell. This minimization and miniaturization enables compact devices capable of rapid signal delivery and detection while reducing the footprint of similar traditional devices (i.e. a standard bench top PCR/QPCR/Fluorometer). Amplification on Chip

In some instances, silicon microelectrode arrays can withstand thermal cycling necessary for PCR. In some applications, on-chip PCR is advantageous because small amounts of target nucleic acids can be lost during transfer steps. In certain embodiments of devices, systems or processes described herein, any one or more of multiple PCR techniques are optionally used, such techniques optionally including any one or more of the following: thermal cycling in the flowcell directly; moving the material through microchannels with different temperature zones; and moving volume into a PCR tube that can be amplified on system or transferred to a PCR machine. In some instances, droplet PCR is performed if the outlet contains a T-junction that contains an immiscible fluid and interfacial stabilizers (surfactants, etc). In certain embodiments, droplets are thermal cycled in by any suitable method.

In some embodiments, amplification is performed using an isothermal reaction, for example, transcription mediated amplification, nucleic acid sequence-based amplification, signal mediated amplification of RNA technology, strand displacement amplification, rolling circle amplification, loop-mediated isothermal amplification of DNA, isothermal multiple displacement amplification, helicase-dependent amplification, single primer isothermal amplification or circular helicase-dependent amplification.

In various embodiments, amplification is performed in homogenous solution or as heterogeneous system with anchored primer(s). In some embodiments of the latter, the resulting amplicons are directly linked to the surface for higher degree of multiplex. In some embodiments, the amplicon is denatured to render single stranded products on or near the electrodes. Hybridization reactions are then optionally performed to interrogate the genetic information, such as single nucleotide polymorphisms (SNPs), Short Tandem Repeats (STRs), mutations, insertions/deletions, methylation, etc. Methylation is optionally determined by parallel analysis where one DNA sample is bisulfite treated and one is not. Bisulfite depurinates unmodified C becoming a U. Methylated C is unaffected in some instances. In some embodiments, allele specific base extension is used to report the base of interest.

Rather than specific interactions, the surface is optionally modified with nonspecific moieties for capture. For example, surface could be modified with polycations, i.e., polylysine, to capture DNA molecules which can be released by reverse bias (−V). In some embodiments, modifications to the surface are uniform over the surface or patterned specifically for functionalizing the electrodes or non electrode regions. In certain embodiments, this is accomplished with photolithography, electrochemical activation, spotting, and the like.

In some applications, a chip may include multiple regions, each region configured to capture DNA fragments of a specific or different size. Chip regions can sometimes vary with respect to voltage, amperage, frequency, pitch, electrode diameter, the depth of the well, or other factors to selectively capture fragments of different sizes in different regions. In some embodiments, each region comprises an array of multiple electrodes.

In various embodiments, devices or regions are run sequentially or in parallel. In some embodiments, multiple chip designs are used to narrow the size range of material collected creating a band pass filter. In some instances, current chip geometry (e.g., 80 um diameter electrodes on 200 um center-center pitch (80/200) acts as 500 bp cutoff filter (e.g., using voltage and frequency conditions around 10 Vpp and 10 kHz). In such instances, a nucleic acid of greater than 500 bp is captured, and a nucleic acid of less than 500 bp is not. Alternate electrode diameter and pitch geometries have different cutoff sizes such that a combination of chips should provide a desired fragment size. In some instances, a 40 um diameter electrode on 100 um center-center pitch (40/100) has a lower cutoff threshold, whereas a 160 um diameter electrode on 400 um center-center pitch (160/400) has a higher cutoff threshold relative to the 80/200 geometry, under similar conditions. In various embodiments, geometries on a single chip or multiple chips are combined to select for a specific sized fragments or particles. For example a 600 bp cutoff chip would leave a nucleic acid of less than 600 bp in solution, then that material is optionally recaptured with a 500 bp cutoff chip (which is opposing the 600 bp chip). This leaves a nucleic acid population comprising 500-600 bp in solution. This population is then optionally amplified in the same chamber, a side chamber, or any other configuration. In some embodiments, size selection is accomplished using a single electrode geometry, wherein nucleic acid of >500 bp is isolated on the electrodes, followed by washing, followed by reduction of the ACEK high field strength (change voltage, frequency, conductivity)in order to release nucleic acids of <600 bp, resulting in a supernatant nucleic acid population between 500-600 bp. In some embodiments, the device is configured to selectively capture nucleic acid fragments between 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, and/or 300-500 bp in length.

In some embodiments, the chip device is oriented vertically with a heater at the bottom edge which creates a temperature gradient column. In certain instances, the bottom is at denaturing temperature, the middle at annealing temperature, the top at extension temperature. In some instances, convection continually drives the process. In some embodiments, provided herein are methods or systems comprising an electrode design that specifically provides for electrothermal flows and acceleration of the process. In some embodiments, such design is optionally on the same device or on a separate device positioned appropriately. In some instances, active or passive cooling at the top, via fins or fans, or the like, provides a steep temperature gradient. In some instances the device or system described herein comprises, or a method described herein uses, temperature sensors on the device or in the reaction chamber monitor temperature and such sensors are optionally used to adjust temperature on a feedback basis. In some instances, such sensors are coupled with materials possessing different thermal transfer properties to create continuous and/or discontinuous gradient profiles.

In some embodiments, the amplification proceeds at a constant temperature (i.e, isothermal amplification).

In some embodiments, the methods disclosed herein further comprise sequencing the nucleic acid isolated as disclosed herein. In some embodiments, the nucleic acid is sequenced by Sanger sequencing or next generation sequencing (NGS). In some embodiments, the next generation sequencing methods include, but are not limited to, pyrosequencing, ion semiconductor sequencing, polony sequencing, sequencing by ligation, DNA nanoball sequencing, sequencing by ligation, or single molecule sequencing.

In some embodiments, the isolated nucleic acids disclosed herein are used in Sanger sequencing. In some embodiments, Sanger sequencing is performed within the same device as the nucleic acid isolation (Lab-on-Chip). Lab-on-Chip workflow for sample prep and Sanger sequencing results would incorporate the following steps: a) sample extraction using ACE chips; b) performing amplification of target sequences on chip; c) capture PCR products by ACE; d) perform cycle sequencing to enrich target strand; e) capture enriched target strands; f) perform Sanger chain termination reactions; perform electrophoretic separation of target sequences by capillary electrophoresis with on chip multi-color fluorescence detection. Washing nucleic acids, adding reagent, and turning off voltage is performed as necessary. Reactions can be performed on a single chip with plurality of capture zones or on separate chips and/or reaction chambers.

In some embodiments, the method disclosed herein further comprise performing a reaction on the nucleic acids (e.g., fragmentation, restriction digestion, ligation of DNA or RNA). In some embodiments, the reaction occurs on or near the array or in a device, as disclosed herein.

### Other Assays

The isolated nucleic acids disclosed herein may be further utilized in a variety of assay formats. For instance, devices which are addressed with nucleic acid probes or amplicons may be utilized in dot blot or reverse dot blot analyses, base-stacking single nucleotide polymorphism (SNP) analysis, SNP analysis with electronic stringency, or in STR analysis. In addition, such devices disclosed herein may be utilized in formats for enzymatic nucleic acid modification, or protein-nucleic acid interaction, such as, e.g., gene expression analysis with enzymatic reporting, anchored nucleic acid amplification, or other nucleic acid modifications suitable for solid-phase formats including restriction endonuclease cleavage, endo- or exo-nuclease cleavage, minor groove binding protein assays, terminal transferase reactions, polynucleotide kinase or phosphatase reactions, ligase reactions, topoisomerase reactions, and other nucleic acid binding or modifying protein reactions.

In addition, the devices disclosed herein can be useful in immunoassays. For instance, in some embodiments, locations of the devices can be linked with antigens (e.g., peptides, proteins, carbohydrates, lipids, proteoglycans, glycoproteins, etc.) in order to assay for antibodies in a bodily fluid sample by sandwich assay, competitive assay, or other formats. Alternatively, the locations of the device may be addressed with antibodies, in order to detect antigens in a sample by sandwich assay, competitive assay, or other assay formats. As the isoelectric point of antibodies and proteins can be determined fairly easily by experimentation or pH/charge computations, the electronic addressing and electronic concentration advantages of the devices may be utilized by simply adjusting the pH of the buffer so that the addressed or analyte species will be charged.

In some embodiments, the isolated nucleic acids are useful for use in immunoassay-type arrays or nucleic acid arrays.

### DEFINITIONS AND ABBREVIATIONS

The articles “a”, “an” and “the” are non-limiting. For example, “the method” includes the broadest definition of the meaning of the phrase, which can be more than one method.

“Vp-p” is the peak-to-peak voltage.

“TBE” is a buffer solution containing a mixture of Tris base, boric acid and EDTA.

“TE” is a buffer solution containing a mixture of Tris base and EDTA.

“L-Histidine buffer” is a solution containing L-histidine.

“DEP” is an abbreviation for dielectrophoresis.

### EXAMPLES

### Example 1

### Isolation of Cell-Free DNA from Plasma Using Disclosed Device and Method V. Conventional Method

QIAGEN® circulating nucleic acid Purification kit (cat#55114) was used to purify 1 ml of plasma from chronic lymphocytic leukemia (CLL) patients, according to manufacturer's protocol. Briefly, incubation of 1 ml plasma with Proteinase K solution was performed for 30 minutes at 60° C. The reaction was quenched on ice and the entire volume was applied to a QIAamp Mini column connected to a vacuum. The liquid was pulled through the column and washed with 3 different buffers (600-750 ul each). The column was centrifuged at 20,000×g, 3 minutes and baked at 56° C. for 10 minutes to remove excess liquid. The sample was eluted in 55 μl of elution buffer with 20,000×g, 1 minute centrifugation. Total processing time was ˜2.5 hours.

The chip die size was 10×12 mm, with 60-80 μm diameter Pt electrodes on 180-200 μm center-to-center pitch, respectively. The array was overcoated with a 5% pHEMA hydrogel layer (spun cast 6000 rpm from Ethanol solution, 12% pHEMA stock from Polysciences). The chip was pretreated using 0.5×PBS, 2V rms, 5 Hz, 15 seconds. The buffer was removed and 25 ₁1.1 of CLL patient plasma was added. DNA was isolated for 3 minutes at 11 V p-p, 10Khz, then washed with 500 μl of TE buffer at a 100 μl/min flow rate, with power ON. The voltage was turned off and the flow cell volume was eluted into a microcentrifuge tube. Total processing time was ˜10 minutes.

The same process can be applied to fresh whole blood without modification. Ability to extract and purify DNA from whole undiluted blood is uniquely enabled by the chip technology disclosed herein.

DNA quantitation was performed on the Qiagen and chip elutes using PicoGreen according to manufacturer's protocol (Life Tech) (Table 2).

Subsequent gel electrophoresis, PCR and Sanger sequencing reactions showed similar performance for both extraction techniques with the chip being able to process whole blood as well as plasma. Mann-Whitney U non-parametric statistical test was also run between DNA amounts isolated from plasma using the Qiagen and chip techniques. There was no statistical difference (p<0.05 two-tailed) using either method of DNA purification.

TABLE 2
DNA purification, chip v. Qiagen
Values are in ng/ml and normalized to original plasma sample
volume for comparison purposes.

| | Chip - | Qiagen - | Chip - |
|---|---|---|---|
| Patient | plasma | plasma | blood |
| normal A | 139 | 39 | 274 |
| normal B | 206 | 80 | 114 |
| normal C | 133 | 32 | 97 |
| TJK 528 | 320 | 547 | 167 |
| TJK 851 | 218 | 393 | 307 |
| TJK 1044 | 285 | 424 | 794 |
| TJK 334 | 261 | 1387 | 666 |
| TJK 613 | 179 | 53 | 257 |
| TJK 762 | 145 | 367 | 314 |
| TJK 847 | 886 | 1432 | 811 |
| TJK 248 | 84 | 119 | 448 |
| TJK 1024 | 302 | 169 | 332 |
| TJK 1206 | 584 | 396 | 1435 |
| TJK 1217 | 496 | 146 | 584 |
| TJK 1262 | 87 | 84 | 1592 |
| TJK 1311 | 119 | 257 | 1825 |

### Example 2

### Isolation and Quantification of Cell-Free DNA from Plasma Obtained from Healthy Patients and Cancer Patients

Plasma samples were obtained from 52 healthy patients and 53 cancer patients. Cell-free DNA fragments greater than 300 bp in size were isolated from the plasma on the devices disclosed herein as described in Example 1. The DNA was labeled using SYBR Green staining and quantified using a CMOS sensor attached to a 4X objective. The amount of cell-free DNA was then quantified for each sample in picograms per microliter of plasma.

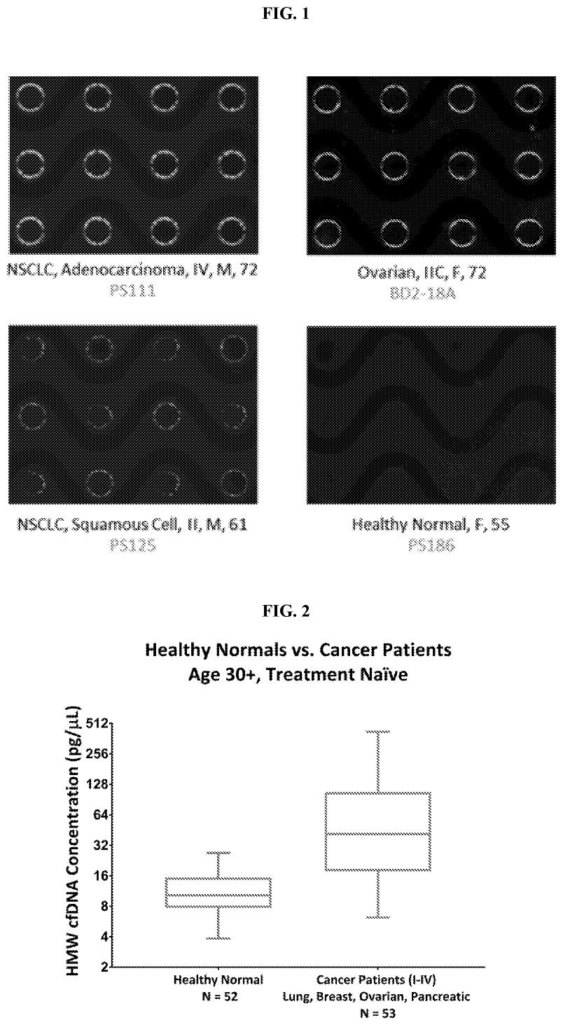

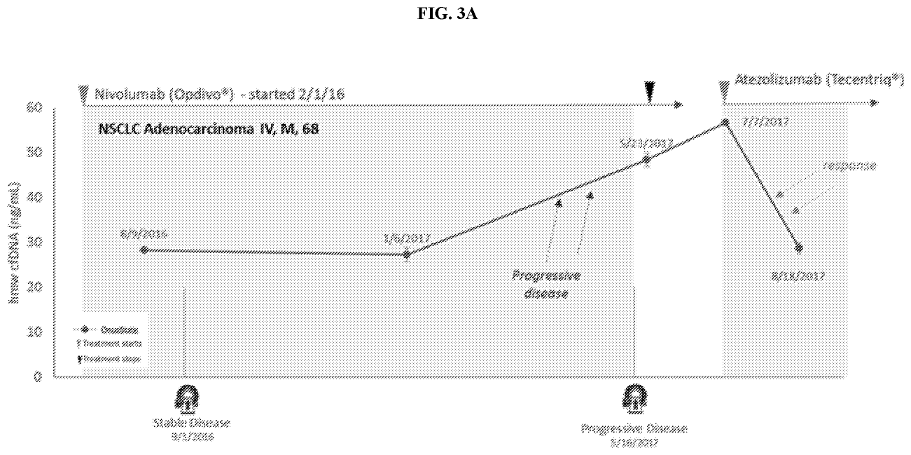

FIG. 1 shows the results of samples from patients with adenocarcinoma, squamous cell cancer, and ovarian cancer, and a healthy control. FIG. 2 shows a comparison of cfDNA concentrations for 52 healthy patients and 53 cancer patients (lung, breast, ovarian, and pancreatic cancers). The results show an increase in cfDNA concentrations for fragments above 300 bp in size in cancer patients as compared to healthy controls.

### Example 3

### Tracking Thereapeutic Response Using Cell-Free DNA Quantification

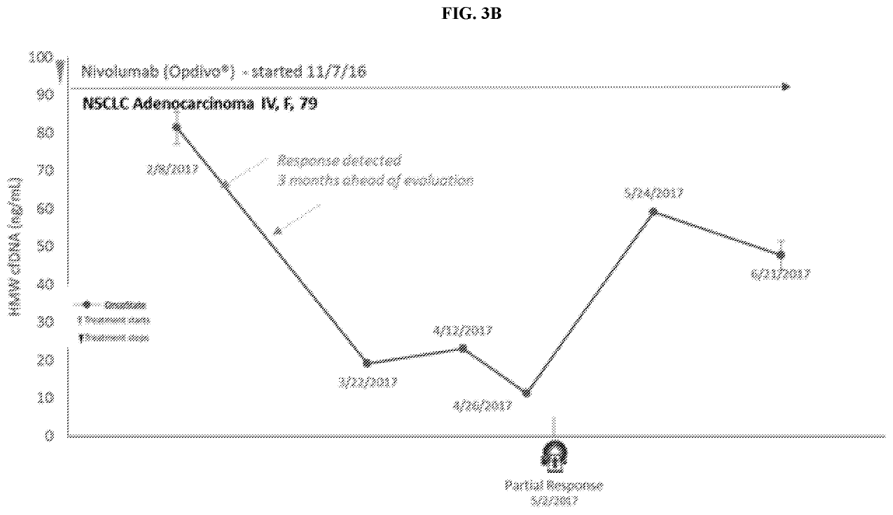

Plasma samples were obtained over the course of treatment for two patients with stage IV adenocarcinoma receiving nivolumab (Opdivo) therapy. The samples were processed and analyzed as described in Example 2. The results are shown in FIG. 3A and FIG. 3B. The patient in FIG. 3A failed to respond to nivolumab treatment, which is shown by a constant level of detected cfDNA after treatment, and then began to show signs of disease progression. The patient was subsequently administered atezolizumab (Tecentriq), to which he responded. The response is indicated by a decrease in cfDNA concentration. The results show that the concentration of cfDNA larger than 300 bp decreases as patients respond to therapy and increases as the disease progresses. The figures show the concentration of cdDNA on the Y axis as ng/mL and the X axis represents time.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for analyzing a biological sample from a subject comprising:
a) capturing a plurality of nucleic acid fragments in the biological sample wherein the plurality of nucleic acid fragments comprises a plurality of sizes, wherein the plurality of sizes comprises nucleic acid fragments at least 250 bp in length;
b) detecting the plurality of nucleic acids; and
c) determining if the subject has a disease or condition based on the detection of nucleic acid fragments at least 250 in length.

2. The method of claim 1, wherein the nucleic acid fragments comprise cell-free DNA fragments and/or exosomes.

3. The method of claim 1 or claim 2, wherein capturing the plurality of nucleic acid fragments comprises selectively capturing nucleic acid fragments between 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, 300-500 bp, 600-700 bp, 700-800 bp, 800-900 bp, 900-1000 bp, 1-2 kbp, 2-3 kbp, 3-4 kbp, 4-5 kbp, 5-6 kbp, 6-7 kbp, 7-8 kbp, 8-9 kbp, and/or 9-10 kbp in length.

4. The method of any of claims 1-3, wherein the disease or condition is cancer.

5. The method of any of claims 1-3, wherein the disease or condition is an inflammatory disease, sepsis, heart disease, an alloimmune condition, or an autoimmune condition.

6. The method of any one of claims 1-5 wherein the detecting comprises quantifying the plurality of nucleic acids.

7. The method of claim 6, wherein quantifying the plurality of nucleic acids comprises quantifying an amount of nucleic acids that have a particular size or size range.

8. The method of claim 7, wherein the at least one size range comprises at least one size range of between 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, 300-500 bp, 600-700 bp, 700-800 bp, 800-900 bp, 900-1000 bp, 1-2 kbp, 2-3 kbp, 3-4 kbp, 4-5 kbp, 5-6 kbp, 6-7 kbp, 7-8 kbp, 8-9 kbp, and/or 9-10 kbp in length.

9. The method of claim 2, wherein quantifying the plurality of nucleic acids comprises quantifying an amount of nucleic acids corresponding to at least one size.

10. The method of claim 9, wherein the at least one size comprises at least one size of any whole number of base pairs between 250 bp and 10 kbp.

11. The method of any of claims 7-10, wherein the method further comprises comparing an amount of nucleic acids corresponding to a first size or size range to a second amount of nucleic acids corresponding to a second size or size range.

12. The method of any of claims 6-11, wherein the method further comprises comparing the amount of nucleic acids to a control.

13. The method of any of claims 7-11, wherein the method further comprises comparing an amount of nucleic acids corresponding to a first size or size range to a control.

14. The method of any of claims 12-13, wherein the control is obtained from the subject at an earlier time, from the subject when the subject is presumed not to have cancer, from a healthy individual, from the subject before undergoing a treatment for cancer, from the subject when the subject was undergoing treatment for cancer, from the subject after undergoing treatment for cancer, or wherein the control is a value determined to be indicative of a subject with cancer or a subject without cancer.

15. The method of any of claims 1-14, wherein determining if the subject has cancer further comprises determining a type of cancer, a stage of cancer, a prognosis, a size of a tumor, a tumor burden, a change in an amount of cancer, a change in tumor size, a change in the number of tumors, a premalignant condition, or a precancerous condition.

16. The method of any one of claims 1-15, wherein capturing the plurality of nucleic acid fragments comprises using an electrode configured to generate an AC dielectrophoretic field.

17. The method of claim 16, wherein the capturing the plurality of nucleic acid fragments comprises using a plurality of electrodes configured to generate a dielectrophoretic low field region and a di electrophoretic high field region.

18. The method of claim 17, wherein the dielectrophoretic low field region is produced using an alternating current having a voltage of 1 volt to 40 volts peak-peak; and/or a frequency of 5 Hz to 5,000,000 Hz, and duty cycles from 5% to 50%.

19. The method of any one of claims 17-18, wherein the dielectrophoretic high field region is produced using an alternating current having a voltage of 1 volt to 40 volts peak-peak;
and/or a frequency of 5 Hz to 5,000,000 Hz, and duty cycles from 5% to 50%.

20. The method of any one of claims 16-19, wherein the plurality of electrodes are configured to selectively capture nucleic acid fragments between 250-600 bp, 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, 300-500 bp, 600-700 bp, 700-800 bp, 800-900 bp, 900-1000 bp, 1-2 kbp, 2-3 kbp, 3-4 kbp, 4-5 kbp, 5-6 kbp, 6-7 kbp, 7-8 kbp, 8-9 kbp, and/or 9-10 kbp in length.

21. The method of any one of claims 16-20, wherein the plurality of electrodes is coated with a hydrogel.

22. The method of claim 21, wherein the hydrogel comprises two or more layers of a synthetic polymer.

23. The method of claim 21 or claim 22, wherein the hydrogel is spin-coated onto the electrodes.

24. The method of any one of claims 21-23, wherein the hydrogel has a viscosity between about 0.5 cP to about 5 cP prior to spin-coating.

25. The method of any one of claims 21-24, wherein the hydrogel has a thickness between about 0.1 microns and 1 micron.

26. The method of any one of claims 16-25, wherein the electrode comprises a material selected from the group consisting of platinum, gold, aluminum, tantalum, gallium arsenide, copper, silver, brass, zinc, tin, nickel, silicon, palladium, titanium, graphite, carbon, and combinations thereof.

27. The method of any one of claims 16-26, wherein the electrode comprises a mixed-metal oxide.

28. The method of claim 27, wherein the mixed-metal oxide is selected from the group consisting of titanium oxide, zirconium.

29. The method of any one of claims 1-28, wherein the method further comprises detecting at least one analyte selected from the group consisting of RNA, nucleosomes, exosomes, extracellular vesicles, proteins, cell membrane fragments, mitochondria or cellular vesicles.

30. The method of any one of claims 1-29, wherein the biological sample comprises a bodily fluid, blood, serum, plasma, urine, saliva, cells, tissue, or a combination thereof.

31. The method of claim 30, wherein the biological sample comprises cells and the method further comprises lysing the cells.

32. The method of any one of claims 1-31, further comprising eluting the captured nucleic acid fragments.

33. The method of any one of claims 1-32, further comprising sequencing the nucleic acid fragments.

34. The method of any one of claims 1-33, wherein an increase in the efficiency of capturing the DNA fragments increases the diagnostic or predictive power or accuracy of the method by at least 10%, 20%, 30%, 40%, 50%, 60%, 70%, 80%, 90%, 100%, 150%, 200%, 300%, 400%, 500%, 1000%.

35. The method of any one of claims 1-34, wherein an increase in the efficiency of capturing the DNA fragments decreases the false positive rate by at least 10%, 20%, 30%, 40%, 50%, 60%, 70%, 80%, 90%, 100%, 150%, 200%, 300%, 400%, 500%, 1000%.

36. The method of any one of claims 1-35, wherein an increase in the efficiency of capturing the DNA fragments decreases the false negative rate by at least 10%, 20%, 30%, 40%, 50%, 60%, 70%, 80%, 90%, 100%, 150%, 200%, 300%, 400%, 500%, 1000%

37. The method of any one of claims 1-36, wherein performance of the method is characterized by an area under the receiver operating characteristic (ROC) curve (AUC) ranging from 0.60 to 0.70, 0.70 to 0.79, 0.80 to 0.89, or 0.90 to 1.00.
